SEQUENCE LISTING (50 sequences)

SEQ ID NO: 1 - protein, 805 aa, Homo sapiens [sequence omitted]

SEQ ID NO: 2 - DNA, 3339 bp, Artificial Sequence
  Synthetic Construct
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 3 - protein, 14 aa, Artificial Sequence
  Synthetic Construct
TGIRDRKKKNKARS

SEQ ID NO: 4 - protein, 14 aa, Artificial Sequence
  Synthetic Construct
TGIRDRQQQNKARS

SEQ ID NO: 5 - protein, 14 aa, Artificial Sequence
  Synthetic Construct
TGIRDRKKQNKARS

SEQ ID NO: 6 - protein, 14 aa, Artificial Sequence
  Synthetic Construct
TGIRDRQKKNKARS

SEQ ID NO: 7 - protein, 14 aa, Artificial Sequence
  Synthetic Construct
TGIRDRKQKNKARS

SEQ ID NO: 8 - protein, 14 aa, Homo sapiens
TGIRDRKQQNKARS

SEQ ID NO: 9 - protein, 14 aa, Artificial Sequence
  Synthetic Construct
TGIRDRQKQNKARS

SEQ ID NO: 10 - protein, 14 aa, Artificial Sequence
  Synthetic Construct
TGIRDRQQKNKARS

SEQ ID NO: 11 - protein, 20 aa, Artificial Sequence
  Synthetic Construct
DISKGENNPGFQNTDDVQTS

SEQ ID NO: 12 - protein, 43 aa, Homo sapiens
TGIRDRKKKNKARSGENPYASIDISKGENNPGFQNTDDVQTSF

SEQ ID NO: 13 - protein, 14 aa, Artificial Sequence
  Synthetic Construct
  VARIANT (7)..(7): independently selected from K and Q amino acids, or modified forms thereof
  VARIANT (8)..(8): independently selected from K and Q amino acids, or modified forms thereof
  VARIANT (9)..(9): independently selected from K and Q amino acids, or modified forms thereof
TGIRDRXXXNKARS

SEQ ID NO: 14 - protein, 14 aa, Artificial Sequence
  Synthetic Construct
  VARIANT (7)..(7): Xaa is dimethyllysine
TGIRDRXKKNKARS

SEQ ID NO: 15 - protein, 14 aa, Artificial Sequence
  Synthetic Construct
  VARIANT (8)..(8): Xaa is dimethyllysine
TGIRDRKXKNKARS

SEQ ID NO: 16 - protein, 14 aa, Artificial Sequence
  Synthetic Construct
  VARIANT (9)..(9): Xaa is dimethyllysine
TGIRDRKKXNKARS

SEQ ID NO: 17 - protein, 16 aa, Artificial Sequence
  Synthetic Construct
  VARIANT (1)..(1): Xaa is absent or is selected from at least one of a proteinaceous moiety comprising from about 1 to about 50 amino acid residues, and a protecting moiety.
  VARIANT (8)..(8): Xaa is independently selected from K and Q amino acids, or modified forms thereof
  VARIANT (9)..(9): Xaa is independently selected from K and Q amino acids, or modified forms thereof
  VARIANT (10)..(10): Xaa is independently selected from K and Q amino acids, or modified forms thereof
  VARIANT (16)..(16): Xaa is absent or is selected from at least one of a proteinaceous moiety comprising from about 1 to about 50 amino acid residues, and a protecting moiety
XTGIRDRXXXNKARSX

SEQ ID NO: 18 - protein, 14 aa, Artificial Sequence
  Synthetic Construct
ARSGENPYASIDIS

SEQ ID NO: 19 - protein, 21 aa, Artificial Sequence
  Synthetic Construct
DISKGENNPGFQNTDDVQTSF

SEQ ID NO: 20 - protein, 19 aa, Artificial Sequence
  Synthetic Construct
ASIDISKGENNPGFQNTDD

SEQ ID NO: 21 - protein, 26 aa, Artificial Sequence
  Synthetic Construct
VQTSFDISKGENNPGFQNTDDVQTSF

SEQ ID NO: 22 - protein, 14 aa, Artificial Sequence
  Synthetic Construct
  VARIANT (1)..(1): Xaa is selected from any small or polar amino acid (preferably, a T or S amino acid), or modified forms thereof;
  VARIANT (5)..(5): Xaa is selected from a D or N amino acid, or modified forms thereof;
  VARIANT (7)..(7): Xaa is selected from K and Q amino acids, or modified forms thereof
  VARIANT (8)..(8): Xaa is selected from K and Q amino acids, or modified forms thereof
  VARIANT (9)..(9): Xaa is selected from K and Q amino acids, or modified forms thereof
  VARIANT (10)..(10): Xaa is selected from any polar amino acid (e.g., an N, K, or D amino acid), or a modified form thereof
  VARIANT (11)..(11): Xaa is selected from a K or Q amino acid, or a modified form thereof
  VARIANT (13)..(13): Xaa is selected from an R, G, or S amino acid, or a modified form thereof
XGIRXRXXXXXAXS

SEQ ID NO: 23 - protein, 13 aa, Artificial Sequence
  Synthetic Construct
  VARIANT (1)..(1): Xaa is absent or is selected from at least one of a proteinaceous moiety comprising from about 1 to about 50 amino acid residues, and/or a protecting moiety
  VARIANT (13)..(13): Xaa is absent or is selected from at least one of a proteinaceous moiety comprising from about 1 to about 50 amino acid residues, and/or a protecting moiety
XIEEQAKTFLDKX

SEQ ID NO: 24 - protein, 32 aa, Artificial Sequence
  Synthetic Construct
IEEQAKTFLDKFNHEAEDLFYQSSLASWNYNT

SEQ ID NO: 25 - protein, 43 aa, Myotis lucifugus
TGIRDRKKKKQAGNEENPYSSVNLSKGENNPGFQNGDDVQTSF

SEQ ID NO: 26 - protein, 13 aa, Artificial Sequence
  Synthetic Construct
STIEEQAKTFLDK

SEQ ID NO: 27 - protein, 43 aa, Felis catus
SGIRNRRKNNQARSEENPYASVDLSKGENNPGFQHADDVQTSF

SEQ ID NO: 28 - protein, 43 aa, Canis lupus familiaris
SGIRNRRKNDQARGEENPYASVDLSKGENNPGFQNVDDAQTSF

SEQ ID NO: 29 - protein, 43 aa, Camelus ferus
TGIRDRRKKKQASTEENPYGSVDLSKGENNSGFQNGDDVQTSF

SEQ ID NO: 30 - protein, 43 aa, Macaca fascicularis
TGIRDRKKKNQARSEENPYASIDINKGENNPGFQNTDDVQTSF

SEQ ID NO: 31 - protein, 10 aa, Artificial Sequence
  Synthetic Construct
DRKKKNKARS

SEQ ID NO: 32 - protein, 10 aa, Myotis lucifugus
DRKKKKQAGN

SEQ ID NO: 33 - protein, 10 aa, Felis catus
NRRKNNQARS

SEQ ID NO: 34 - protein, 10 aa, Canis lupus familiaris
NRRKNDQARG

SEQ ID NO: 35 - protein, 10 aa, Camelus ferus
DRRKKKQAST

SEQ ID NO: 36 - protein, 10 aa, Macaca fascicularis
DRKKKNQARS

SEQ ID NO: 37 - protein, 11 aa, Artificial Sequence
  Synthetic Construct
  LIPID (1)..(1): MYRISTATE
RRTSRRKRAKV

SEQ ID NO: 38 - protein, 15 aa, Artificial Sequence
  Synthetic Construct
  VARIANT (1)..(1): Xaa is a fluorescein tagged N-terminal aminohexanoic acid (Ahx)
XRDRKKKNKARSGEN

SEQ ID NO: 39 - protein, 15 aa, Artificial Sequence
  Synthetic Construct
  VARIANT (8)..(8): Xaa is dimethyllysine
QAKTFLDXFNHEAED

SEQ ID NO: 40 - protein, 14 aa, Artificial Sequence
  Synthetic Construct
  VARIANT (7)..(7): Xaa is dimethyllysine
TGIRDRXKKNKARS

SEQ ID NO: 41 - protein, 14 aa, Artificial Sequence
  Synthetic Construct
  VARIANT (8)..(8): Xaa is dimethyllysine
TGIRDRKXKNKARS

SEQ ID NO: 42 - protein, 14 aa, Artificial Sequence
  Synthetic Construct
  VARIANT (9)..(9): Xaa is dimethyllysine
TGIRDRKKXNKARS

SEQ ID NO: 43 - protein, 14 aa, Artificial Sequence
  Synthetic Construct
  VARIANT (7)..(7): Xaa is dimethyllysine
  VARIANT (9)..(9): Xaa is dimethyllysine
TGIRDRXKXNKARS

SEQ ID NO: 44 - protein, 14 aa, Artificial Sequence
  Synthetic Construct
  LIPID (1)..(1): MYRISTATE
TGIRDRKKKNKARS

SEQ ID NO: 45 - DNA, 22 bp, Artificial Sequence
  Synthetic Construct
ccgcaaggttcttcttcgtaag

SEQ ID NO: 46 - DNA, 25 bp, Artificial Sequence
  Synthetic Construct
tgctatgtttagtgttccagttttc

SEQ ID NO: 47 - DNA, 25 bp, Artificial Sequence
  Synthetic Construct
  variation (1)..(1): 5′ is Hexachlorofluorescein (HEX) modified
  variation (25)..(25): 3′ is Black Hole Quencher-1 (BHQ-1) modified
aaggatcagtgccaagctcgtcgcc

SEQ ID NO: 48 - protein, 6 aa, Artificial Sequence
  Nuclear localisation sequence
RKKKNK

SEQ ID NO: 49 - protein, 32 aa, Artificial Sequence; identical to SEQ ID NO: 24
  ACE2-01

SEQ ID NO: 50 - protein, 13 aa, Artificial Sequence
  ACE2-02
STIEEQAKTFLDK

(19) United States
(12) Patent Application Publication
(10) Pub. No.: US 2024/0352072 A1
(43) Pub. Date: Oct. 24, 2024

(54) NOVEL COMPOSITIONS AND METHODS FOR TREATING CORONAVIRUS INFECTIONS

(71) Applicant: The Council of the Queensland Institute of Medical Research, Herston, Queensland (AU)

(72) Inventor: Sudha RAO, Herston (AU)

(21) Appl. No.: 18/287,793

(22) PCT Filed: Apr. 20, 2022
(86) PCT No.: PCT/AU2022/050363

§ 371 Date: Oct. 20, 2023

(30) Foreign Application Priority Data
Apr. 20, 2021 (AU) 2021901169
Feb. 18, 2022 (AU) 2022900358

(51) Int. Cl. C07K 7/08; A61K 38/00; A61K 45/06; A61P 31/14; C07K 14/005

(52) U.S. Cl. CPC C07K 7/08; A61K 45/06; A61P 31/14; C07K 14/005; A61K 38/00; C12N 2770/20022

(57) ABSTRACT

Disclosed are compositions and methods suitable for treating coronavirus infections. More particularly, disclosed is a proteinaceous agent that prevents or inhibits the replication of a SARS-CoV virus, including a SARS-CoV-2 virus. Also disclosed is the use of these agents and molecules for treating or preventing a coronavirus infection (including a SARS-CoV-2 infection) in a subject.

### RELATED APPLICATIONS

This application claims priority to Australian Provisional Application Nos. 2021901169 filed on 20 Apr. 2021 and 2022900358 filed on 18 Feb. 2022, both entitled “NOVEL COMPOSITIONS AND METHODS FOR TREATING CORONAVIRUS INFECTIONS”, the entire contents of which are incorporated herein by reference.

### FIELD OF THE INVENTION

This invention relates generally to methods and compositions for treating coronavirus infections. More particularly, the present invention relates to proteinaceous agents that prevent or inhibit the replication of a SARS-CoV virus, including a SARS-CoV-2 virus. The present invention further relates to the use of these agents and molecules for treating or preventing a coronavirus infection in a subject.

### BACKGROUND OF THE INVENTION

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Coronaviruses are enveloped RNA viruses that infect mammals and birds. The severe acute respiratory syndrome (SARS) and the Middle East respiratory syndrome (MERS) are both members of the genus Betacoronavirus, and responsible for hundreds of deaths in Asia and the Middle East, respectively. The late 2019 emergence in China of the novel, SARS-coronavirus 2 (SARS-CoV-2) pathogen, with rapid human to human transmission and international spread, poses an immediate global health emergency. In response, a global effort for effective treatments is underway following the World Health Organisation's (WHO) declaration of a pandemic, based on the substantial number of cases of the SARS-CoV-2 illness (COVID-19) in over 110 countries and territories in only a few months, and with a sustained risk of further global spread. There is an urgent need for both an effective coronavirus vaccine to prevent the spread of this virus and in parallel, novel therapeutic strategies to reduce the global mortality numbers, which stands currently at just around 5,000 (March 2020). This is compounded by the fact that there is no immunity in the community against this virus. Furthermore, the elderly and the sick are the most at risk with mortality due, mostly, to the weakening of their immune system.

Identifying therapeutic strategies are considered to be the fastest means of addressing this pandemic. One strategy being adopted in treatment developments is combining know drugs for other pathogenic diseases to determine any effectiveness in treating coronavirus infection. Advanced studies are progressing using combinations including an HIV drug and chloroquine (an antimalaria drug, now rarely used as the malaria pathogen has become resistant to it); and between two existing drugs lopinavir and ritonavir (see, Cao et al, 2020). However, due to unintended side effects, in addition to a lack of substantial evidence to demonstrate their efficacy in treating coronavirus infection, there is still a clear unmet clinical need to develop new treatment options specifically for coronavirus.

The coronaviruses are a virus family grouped into four genera, being the alphacoronavirus, betacoronavirus (β-CoVs), gammacoronavirus, and deltacoronavirus. The alphacoronaviruses and betacoronaviruses infect a wide range of species, including humans. In this regard, the β-CoVs that are of particular clinical importance in humans include OC43 and HKU1 of the A lineage, Severe Acute Respiratory Syndrome coronavirus (SARS-CoV) and SARS-CoV-2 (which causes the disease COVID-19) of the B lineage, and Middle Eastern Respiratory Syndrome-related coronavirus (MERS-CoV) of the C lineage.

### SUMMARY OF THE INVENTION

The present invention arises at least in part from the unexpected realisation by the present inventors that host ACE2 protein nuclear localisation is an important function in the SARS-CoV virus infection of a host cell. Furthermore, the nuclear localisation of host ACE2 protein provides a molecular mechanism that can be disrupted in order to prevent SARS-CoV virus replication in the host cell. These realisations have been reduced to practice in novel compositions and methods for treating or preventing coronavirus infections (particularly, SARS-CoV infections).

Accordingly, in one aspect, the present invention provides isolated or purified proteinaceous molecules reducing or inhibiting nuclear localisation of the ACE2 protein. These molecules generally comprise, consist, or consist essentially of an amino acid sequence represented by Formula I:

(Formula I)
TGIRDRX₁X₂X₃NKARS

wherein X₁, X₂, and X₃ are independently selected from K and Q amino acids, or modified forms thereof [SEQ ID NO: 13].

In some preferred embodiments, each of X₁, X₂, and X₃ are K amino acid residues.

In some particularly preferred embodiments the proteinaceous molecule comprises, consists or consists essentially of the amino acid sequence TGIRDRKKKNKARS [SEQ ID NO: 3].

In some alternative embodiments, the proteinaceous molecule comprises, consists, or consists essentially of the amino acid sequence TGIRDRQQQNKARS [SEQ ID NO: 4]. In some alternative embodiments, the proteinaceous molecule comprises, consists, or consists essentially of the amino acid sequence TGIRDRKKQNKARS [SEQ ID NO: 5]. In some alternative embodiments, the proteinaceous molecule comprises, consists, or consists essentially of the amino acid sequence TGIRDRQKKNKARS [SEQ ID NO: 6]. In some other embodiments, the proteinaceous molecule comprises, consists, or consists essentially of the amino acid sequence TGIRDRKQKNKARS [SEQ ID NO: 7]. In some alternative embodiments, the proteinaceous molecule comprises, consists, or consists essentially of the amino acid sequence TGIRDRKQQNKARS [SEQ ID NO: 8]. In some other embodiments, the proteinaceous molecule comprises, consists, or consists essentially of the amino acid sequence TGIRDRQKQNKARS [SEQ ID NO: 9]. In some alternative embodiments, the proteinaceous molecule comprises, consists, or consists essentially of the amino acid sequence TGIRDRQQKNKARS [SEQ ID NO: 10].

In illustrative examples, the proteinaceous molecules comprise, consist, or consist essentially the amino acid sequence TGIRDRKKKNKARS [SEQ ID NO: 3]. In some of the same embodiments and some alternative embodiments, one, two, or each of X₁, X2, and X₃ are methylated K (lysine) residues. Accordingly, in some embodiments the proteinaceous molecule comprises, consists, or consists essentially of an amino acid sequence selected from the group comprising: TGIRDRK(Me2)KKNKARS [SEQ ID NO: 14]; TGIRDRKK(Me2)KNKARS [SEQ ID NO: 15]; and TGIRDRKKK(Me2)NKARS [SEQ ID NO: 16]. In some of the same embodiments and or some alternative embodiments, one, two, or each of X₁, X₂, and X₃ are acetylated K residues.

In some embodiments, the proteinaceous molecule comprises, consists essentially, or consists of an amino acid sequence which is represented by Formula II:

(Formula II)
Z₁TGIRDRX₁X₂X₃NKARSZ₂

wherein X₁, X₂, and X₃ are as broadly defined above;
Z₁ is absent or is selected from at least one of a proteinaceous moiety comprising from about 1 to about 50 amino acid residues, and a protecting moiety; and
Z₂ is absent or is selected from at least one of a proteinaceous moiety comprising from about 1 to about 50 amino acid residues [SEQ ID NO: 17].

In some of the same embodiments and some alternative embodiments, the proteinaceous molecule comprises a ubiquitination site. In some preferred embodiments the ubiquitination site is located in the C-terminal tail region (i.e., amino acid residues 763-805 of the full-length human ACE2 sequence as set forth in SEQ ID NO: 1). In some embodiments, the ubiquitination site comprises the amino acid residue K788.

By way of an illustrative example the proteinaceous molecule may comprise, consist, or consist essentially of the amino acid sequence DISKGENNPGFQNTDDVQTS [SEQ ID NO: 11].

In some of the same embodiments and some other embodiments, the proteinaceous molecules may comprise an amino acid sequence that corresponds to both a methylation site and a ubiquitin site. For example, the proteinaceous molecule may comprise, consists, or consists essentially of the amino acid sequence TGIRDRKKKNKARSGENPYASIDISKGENNPGFQNTDDVQTSF [SEQ ID NO: 12].

In some embodiments, the proteinaceous molecules comprise, consist, or consist essentially of, an amino acid sequence corresponding to the C-terminal tail region sequence of the ACE2 polypeptide that intervenes the methylation sites and the ubiquitination site. By way of an example, the ACE2 peptide may comprise, consist or consist essentially of an amino acid sequence that corresponds to residues 774-787 of the full-length human ACE2 protein (i.e., ARSGENPYASIDIS) [SEQ ID NO: 18].

In another related aspect, the present invention provides a composition for treating or preventing a coronavirus infection, comprising an agent selected from a proteinaceous molecule and a pharmaceutically acceptable carrier or diluent, wherein the proteinaceous molecule as described above and/or elsewhere herein.

In some embodiments of this type, the composition comprises a proteinaceous molecule comprising, consisting, or consisting essentially of a first amino acid sequence which is represented by Formula I (SEQ ID NO: 13) or Formula II (SEQ ID NO: 17), and second amino acid sequence which identified by SEQ ID NO: 11.

In some embodiments, the first amino acid sequence and the second amino acid sequence are located in the same polypeptide. Alternatively, in some embodiments the first amino acid sequence and the second amino acid sequence are present on different polypeptides.

In some of the same embodiments and some other embodiments, the composition comprises at least one anti-viral agent.

In yet another aspect, the present invention provides methods for preventing or reducing coronavirus replication in a host cell, the method comprising contacting the cell with a proteinaceous molecule as described above and/or elsewhere herein for a time and under conditions sufficient to prevent or reduce coronavirus entry in the cell.

In still yet another aspect, the present invention provides a method for treating or preventing a coronavirus infection (e.g., COVID-19) in a subject, the method comprising administering to the subject an effective amount of a proteinaceous molecule described above and/or elsewhere herein. Preferably, the proteinaceous molecule has an amino acid sequence as set forth in Formula I and/or Formula II.

In some preferred embodiments, the coronavirus is a betacoronavirus. Typically, the coronavirus is selected from the group comprising SARS-CoV and SARS-CoV-2. In this regard, in some embodiments the coronavirus is SARS-CoV-2. In some preferred embodiments, the subject is a human.

In yet another aspect, the present invention provides the use of a proteinaceous molecule as described above and/or elsewhere herein, for therapy.

In some embodiments, the methods comprise concurrently, sequentially, or subsequently administering to the subject an antiviral agent.

In some embodiments of this type, the antiviral agent is selected from the group comprising hydroxychloroquine, chloroquine, lopinavir, ritonavir, favipiravir, and remdesivir. In some of the same embodiments and some other embodiments, the antiviral agent comprises an IFN-γ polypeptide.

In another aspect, the present invention provides a pharmaceutical composition that comprises, consists, or consists essentially of an ACE2 peptide as described above and/or elsewhere herein and a pharmaceutically acceptable excipient, carrier and/or diluent. In some embodiments the pharmaceutical composition also comprises an antiviral agent.

In yet another aspect, the present invention provides a method for reducing ACE2 nuclear localisation in a cell, the method comprising contacting the cell with an agent selected from a proteinaceous molecule or composition as described above or elsewhere herein for a time and under conditions sufficient to reduce nuclear localisation in the cell.

In still yet another aspect, the present invention provides a method for reducing or preventing the binding of an ACE2 polypeptide to an IMPα polypeptide, the method comprising contacting the cell with an agent selected from a proteinaceous molecule or composition as described above or elsewhere herein for a time and under conditions sufficient to reduce, prevent inhibit the binding of an ACE2 polypeptide to an IMPα polypeptide.

In some embodiments, when the proteinaceous molecules of the invention are administered to a subject, inflammation (e.g., lung inflammation) is reduced in the subject. In some embodiments, the level of cells expressing CD3+ is increased in the lung of the subject. In some of the same embodiments and some different embodiments, the level of cells expressing perforin is increased in the lung of the subject.

### BRIEF DESCRIPTION OF THE FIGURES

Examples of the present invention will now be described with reference to the accompanying figures, in which:

FIG. 1 shows that LSD1 and ACE2 associate as a complex on cell surface in SARS-CoV-2 susceptible cells. (A) Representative images of CaCo2 cells imaged with the ASI digital pathology system. Cells are either permeabilized (intracellular) or not permeabilized (surface) and stained for expression of ACE2, LSD1 and TRMPSS2. Scale bar represents 10 mm. (B) Dot graphs display the nuclear fluorescence intensity in Caco-2 cells for ACE2, LSD1 and TRMPSS2 from (A). The PCC(r) was calculated for LSD1 and ACE2, (n=20 individual cells). −1=inverse of colocalization; 0=no colocalization; +1=perfect colocalization. (C) Representative FACS plot showing the cell surface and intracellular expression of ACE2 and LSD1 in Caco-2 cells. The numbers in each quadrant indicate the percentage of the total cell population, which also shown in dot plot (D). Data in dot plot represent two independent biological replicates. (E) Representative image of MRC5 cells imaged with the ASI digital pathology system, that are either permeabilized (intracellular) or not permeabilized (surface) and stained for expression of ACE2, LSD1 and TRMPSS2. Scale bar represents 10 mm. (F) Dot graphs displays the nuclear fluorescence intensity in MRC5 cells for ACE2, LSD1 and TRMPSS2 from (E). >50 cells counted per group. Data represent mean±SE. Mann-Whitney-test. **p<0.01, ***p<0.001, ****p<0.0001 denote significant differences. n.s. denotes non-significant.

FIG. 2 provides graphical representation showing LSD1 and ACE2 have increased association on the cell surface in SARS-CoV-2 infected cells. (A, B) Representative image of Caco-2-SARS-CoV-2 infected cells imaged with the ASI digital pathology system (MRC5/Caco-2 uninfected not show) are shown, cells were either (A) permeabilized (intracellular) with 0.5% Triton X-100 for 15 minutes or (B) not permeabilized (surface) and stained for with primary antibodies against ACE2, LSD1 and SARS-CoV-2 nucleocapsid protein. Dot graphs displaying the nuclear fluorescence intensity in Caco-2 cells for ACE2, LSD1 and SARS-CoV-2 from (A). (C) Representative image of Caco-2 or Caco-2-SARS-CoV-2 infected cells imaged with the ASI digital pathology system are shown, cells were either permeabilized (intracellular) with 0.5% Triton X-100 for 15 minutes or not permeabilized (surface) and stained for with primary antibodies against ACE2, LSD1 and SARS-CoV-2 SPIKE protein. Dot graphs displaying the nuclear fluorescence intensity in Caco-2 cells for ACE2, LSD1 and SARS-CoV-2 from (C). The PCC(r) was calculated for LSD1 and ACE2 or LSD1 and SARS-CoV-2, (n=20 individual cells). −1=inverse of colocalization; 0=no colocalization; +1=perfect colocalization. (D) qRT-PCR analysis to detect the growth kinetics of SARS-CoV-2 in Caco-2 and MRC5 culture supernatant at indicated time points after viral infection. The dotted line indicates the limit of detection. (E) FACS analysis of the expression of SARS-CoV-2 nucleocapsid protein, cell surface ACE2 and intracellular LSD1 in Caco-2 cells after 48 hours post-infection. The unit of y axis indicates the percentage of the total cell population. Data represent mean±SD, n=2. (F) Representative image of Caco-2 or Caco-2-SARS-CoV-2 infected cells imaged with the ASI digital pathology system are shown, cells were permeabilized (intracellular) with 0.5% Triton X-100 for 15 minutes and stained for with primary antibodies against H3k9me2 and H3k4me2. (G) Dot graphs displaying the nuclear fluorescence intensity in CaCo2 cells for ACE2, LSD1 and SARS-CoV-2 from (F). (H, I) Representative image of CaCo2 or CaCo2-SARS-CoV-2 infected cells imaged with ASI digital pathology system are shown, cells were either (H) not permeabilized (surface) or (I) permeabilized (intracellular) with 0.5% Triton X100 for 15 minutes or and stained for with primary antibodies against SETDB1, G9A and ACE2. (J) Dot graphs displaying the nuclear fluorescence intensity in Caco-2 cells for ACE2, LSD1 and SARS-CoV-2 from (H, I). >50 cells counted per group. Data represent mean±SE. Mann-Whitney-test. **p<0.01, ***p<0.001, ****p<0.0001 denote significant differences. n.s. denotes non-significant. Scale bars represents 12 mm.

FIG. 3 provides graphical representations showing LSD1 directly interacting with the ACE2 cytoplasmic tail that harbours high affinity LSD1 demethylation domain. (A) The dimer structure of ACE2 is depicted as a schematic. We have identified using high resolution bioinformatic tools in the C-terminal flexible domain sequence which is predicted to be an NLS that binds IMPa. This motif also contains 3 lysine residues (in red) that are high probability de-methylation targets for LSD1 catalytic activity, with an SVM probability of 0.72 or higher. (B) Microscale thermophoresis was carried out to determine the binding between LSD1 and ACE2 via the C-terminal tail region. Analysis revealed a 1 to 1 binding affinity, indicating strong interaction between LSD1 and ACE2. (C, D) Representative image of Caco-2 cells imaged with the ASI digital pathology system are shown. Caco-2 cells were treated with vehicle control or 200 mM of phenelzine and imaged with the ASI digital pathology system are shown, cells were either (C) permeabilized (intracellular) with 0.5% Triton X-100 for 15 minutes or (D) non-permeabilized (surface) and stained for with primary antibodies against ACE2, LSD1 and SARS-CoV-2 nucleocapsid protein. Scale bar represents 12 mm. (E, F) Dot graphs displays the nuclear fluorescence intensity in Caco-2 cells for ACE2, LSD1 and SARS-CoV-2 from (C, D, respectively). >50 cells counted per group. Data represent mean±SE. Mann-Whitney-test. **p<0.01, ***p<0.001, ****p<0.0001 denote significant differences. n.s. denotes non-significant. The PCC(r) was calculated for LSD1 and ACE2 (n=20 individual cells). −1=inverse of colocalization; 0=no colocalization; +1=perfect colocalization.

FIG. 4 provides a graphical representation of a global transcript analysis. Caco-2 cells were treated with Phenelzine, GSK or L1 and global RNA transcriptome analysis shows that key anti-viral and transcription processes are impacted. The heat map above focuses on a DEGs list related to ISG, IFN-I, cytokine/chemokine activity, and viral entry, nuclear import/RNA synthesis, translation and replication. The heatmap graph depicts the log 2 (fold change) of DEGs of inhibition treated compared with control cells. Those selected DEGs have a log 2 (fold change) of more than 1 and FDR value of less than 0.01.

FIG. 5 shows a graphical representation of the interplay of intracellular ACE2 in infected cells. (A) Representative image of Caco-2 or MRC5 SARS-CoV-2 infected cells are depicted. Scale bar represents 15 mm. (B) Cells were permeabilized and imaged with the ASI digital pathology system are shown, cells were stained for with primary antibodies against SARS-CoV-2 (nucleocapsid), ACE2 and LSD1. Dot graphs display the nuclear fluorescence intensity in Caco-2 cells for ACE2, LSD1. 20 or more cells counted per group. (C) Cells were not permeabilized to track surface expression and stained for with primary antibodies against ACE2 and LSD1. Dot graphs display the nuclear fluorescence intensity in Caco-2 cells for ACE2, LSD1. 20 or more cells counted per group. Data represent mean±SEM. Mann-Whitney-test. *p<0.0181, **p<0.01, ***p<0.001, ****p<0.0001 denote significant differences. n.s. denotes non-significant. The nuclear/cytoplasmic fluorescence ratio (Fn/c) using the equation: Fn/c=(Fn−Fb)/(Fc−Fb), where Fn is nuclear fluorescence, Fc is cytoplasmic fluorescence, and the dotted line indicates background fluorescence. The Mann-Whitney nonparametric test (Graph Pad Prism, GraphPad Software, San Diego, CA) was used to determine significant differences between datasets.

FIG. 6. (A) Representative image of Caco-2 cells imaged with the ASI digital pathology system are shown, that are not permeabilized (surface stain), treated with vehicle control or 25 mM/50 mM of ACE2 novel peptide inhibitor (tagged with FAM5) and stained for cell surface expression of ACE2, LSD1. (B) Dot graphs displays the nuclear fluorescence intensity in Caco-2 cells for ACE2 and LSD1 from (A). (C) Representative image of Caco-2 cells imaged with the ASI digital pathology system are shown, permeabilized with 0.5% Triton X-100, treated with vehicle control or 25 mM/50 mM of ACE2 novel peptide inhibitor (tagged with FAM5) and stained for cell surface expression of ACE2, LSD1. (D) Dot graphs displays the nuclear fluorescence intensity in Caco-2 cells for ACE2 and LSD1 from (C). The nuclear/cytoplasmic fluorescence ratio (Fn/c) using the equation: Fn/c=(Fn−Fb)/(Fc−Fb), where Fn is nuclear fluorescence, Fc is cytoplasmic fluorescence, and Fb is background fluorescence. The Mann-Whitney nonparametric test (GraphPad Prism, GraphPad Software, San Diego, CA) was used to determine significant differences between datasets. (E) Representative image of Caco-2 cells imaged with the ASI digital pathology system are shown, that are not permeabilized (surface stain), treated with vehicle control or 25 mM/50 mM of ACE2 novel peptide inhibitor (with no TAG) and stained for cell surface expression of ACE2, LSD1. (F) Dot graphs display the nuclear fluorescence intensity in Caco-2 cells for ACE2 and LSD1 from (E). >50 cells counted per group. Data represent mean±SE. Mann-Whitney-test. **p<0.01, ***p<0.001, ****p<0.0001 denote significant differences. n.s. denotes non-significant. The PCC(r) was calculated for LSD1 and ACE2, (n=20 individual cells). −1=inverse of colocalization; 0=no colocalization; +1=perfect colocalization. (G) Representative image of Caco-2 cells imaged with the ASI digital pathology system are shown, treated with a novel peptide inhibitor (with FAM5 tag) to demonstrate stability of the novel ACE2 inhibitor over time in media and target cells. Scale bar represents 10 mm.

FIG. 7 shows graphical representations of ACE2 peptide inhibitor effect on nucleocapsid and Spike protein of SARS-Cov-2. (A) Representative image of Caco-2-SARS-CoV-2 infected cells treated with phenelzine (P400 mM), GSK (G400 mM), L1 (50 mM) or P604 (ACE2 peptide 50 mM) imaged with the ASI digital pathology system are shown, Scale bar represents 15 mm. (B) Cells were permeabilized with 0.5% Triton X-100 for 15 minutes and stained for with primary antibodies against ACE2, TMPRSS2 and SARS-CoV-2 Spike Protein. Dot graphs displays the nuclear fluorescence intensity in Caco-2 cells for ACE2, TMPRSS2 and SARS-CoV-2 Spike Protein. The PCC(r) was calculated for ACE2 and SARS-CoV-2. (C) Representative image of Caco-2-SARS-CoV-2 infected cells treated with phenelzine (P400 mM), GSK (G400 mM), L1 (50 mM) or P604 (ACE2 peptide 50 mM) imaged with the ASI digital pathology system are shown, Scale bar represents 15 mm. (D) Cells were permeabilized with 0.5% Triton X-100 for 15 minutes and stained for with primary antibodies against ACE2, TMPRSS2 and SARS-CoV-2 nucleocapsid protein. Dot graphs displays the nuclear fluorescence intensity in Caco-2 cells for ACE2, TMPRSS2 and SARS-CoV-2 nucleocapsid. >50 cells counted per group. Data represent mean±SEM. Mann-Whitney-test. **p<0.01, ***p<0.001, ****p<0.0001 denote significant differences. n.s. denotes non-significant.

FIG. 8. Caco-2 or MRC5 cells were transfected with either VO or LSD1 WT plasmids. Cells were either permeabilized (intracellular) with 0.5% Triton X-100 for 15 minutes and stained for with primary antibodies against (A) ACE2 and (B) LSD1 and imaged with the ASI digital pathology system. Bar graphs displays the overall mean fluorescence intensity for LSD1 and Caco-2 cells >20 cells counted per group. Data represent mean±SE. Mann-Whitney-test. **p<0.01, ***p<0.001, ****p<0.0001 denote significant differences. n.s. denotes non-significant.

FIG. 9 provides graphical representations of the ACE2 and spike protein interaction. (A) Structure of ACE2 bound to the SARS-CoV-2 spike domain (PDB 6M17). Binding of ACE2 and the spike domain involves a Lys31 (ACE2) and Gln493 (spike) interaction. ACE2 is shown in yellow in cartoon mode, and spike domain in grey. Residues are shown in stick format. Methylation of ACE2 Lys31 (right panel) would disrupt this interaction. (B) Structure of ACE2 bound to the SARS-CoV-2 spike protein as depicted in panel A. Also depicted are the two peptide inhibitors targeting this region (ACE2-01, ACE2-02) and the interaction with the SARS-CoV-2 spike protein. (C) Cell proliferation analysis of Caco-2 control and ACE2-01/ACE2-02-treated cells over a 96-hour period. Proliferation was analysed using WST-1 reagent and absorbance read after 2-hour incubation. The graph depicts relative cell proliferation from three replicates expressed as a percentage of control cells (untreated, 0 hours). Statistical significance was calculated using one-way ANOVA at each time point. (D) Schematic of SARS-CoV-2 infection. Caco-2 cells were seeded for 24 hours and then infected with SARS-CoV-2 at MOI 1.0 in the presence of ACE2 peptide inhibitors (ACE2-01 or ACE2-02) for 1 hour. The virus inoculum was removed and inhibitor-containing medium was added. Then, cell culture supernatants were collected at 0 or 48 hpi and infected cells were harvested at 48 hpi. Antiviral activity was assessed with three viral assays: SARS-CoV-2 qRT-PCR, median tissue culture infective dose assay (TCID₅0), and viral spike protein quantified by digital pathology (ASI system). (E) qRT-PCR analysis to detect replicates of SARS-CoV-2 RNA in Caco-2 culture supernatants and infected cells at the indicated time points post-infection. Relative infection was normalized to the uninfected control. Data represent mean±SEM, n=3. One-way ANOVA, ****p<0.0001 denotes significant differences. (F) TCID₅₀ assay to measure infectious viral titers in the culture supernatants of infected cells. Data represent mean±SEM, n=3. One-way ANOVA, ** p<0.01 denotes significant differences. (G) Dot plot quantification of the fluorescence intensity (cell surface) of SARS-CoV-2 spike and ACE2 in SARS-CoV-2-infected Caco-2 cells with ACE2-01 or ACE2-02 treatment. (H) Dot plot quantification of the fluorescence intensity (intracellular) of SARS-CoV-2 spike and ACE2 in SARS-CoV-2-infected Caco-2 cells with ACE2-01 or ACE2-02 treatment. >50 cells were analysed in each group and were quantified by digital pathology (ASI system). The PCC was calculated for colocalization (n=20 cells were analysed). Mann-Whitney test: * p<0.05, **p<0.01, ****p<0.0001 denote significant differences. (1) Duolink® proximity ligation assay measurements of protein interactions were performed on unpermeabilized Caco-2 cells infected with SARS-CoV-2 and treated with control, GSK, or ACE2-01 or ACE2-01 peptide inhibitors. The Duolink® assay produces a single bright dot per interaction within the cell. Representative images (left) are shown for ACE2 and SARS-CoV-2 Spike Duolink®. PLA signal intensity of the Duolink® assay (right) is shown for average dot intensity (single Duolink® dot). Data represent n=20 cells, with significant differences calculated with Kruskal-Wallis ANOVA (* p<0.05, ****p<0.0001). Representative images are shown with 10 μM scale bar in orange.

FIG. 10. (A) The receptor-binding domain (RBD) sequence of SARS-CoV-2 showing the critical residue (Q; glutamine 493) that binds to ACE2 lysine 31 and the conservation of this sequence in different species. In silico prediction gave a probability of 0.7 of a methylation/demethylation signature at lysine 31. (B) LSD1 inhibition reduces ACE2 demethylation at lysine 31, recombinant LSD1 protein alone or pre-incubated with di-methylated ACE2 peptide.

FIG. 11 provides a graphical representation of a mutagenesis study of ACE2 peptide inhibitors. (A) ACE2-01 Alanine walk peptides were created via an alanine substitution along the length of the peptide. Each peptide was then used to pre-treat CaCo2 cells, followed by treatment with SARS-CoV-2 Spike protein. Samples were then stained with antibodies specific for SARS-CoV-2 spike protein and visualized using an ASI high resolution microscopy. Fluorescent intensities of spike protein on non-permeabilized cells were quantified using ASI Digital pathology software. Data represent n>300 cells per a group. All samples below the red line represent significant reduction in spike protein on cell surface (calculated with Kruskal-Wallis ANOVA with p score of **** (p<0.0001, NS denotes no-significance). (B) ACE2-02 Alanine walk peptides were created via an alanine substitution along the length of the peptide. Each peptide was then used to pre-treat CaCo2 cells, followed by treatment with SARS-CoV-2 Spike protein. Samples were then stained with antibodies specific for SARS-CoV-2 spike protein and visualized using an ASI high resolution microscopy. Fluorescent intensities of spike protein on non-permeabilized cells were quantified using ASI Digital pathology software. Data represent n>300 cells per a group. All samples below the red line represent significant reduction in spike protein on cell surface (calculated with Kruskal-Wallis ANOVA with p score of **** (p<0.0001, NS denotes no-significance).

FIG. 12 provides a graphical representation of P604 ACE2 peptide disrupting nuclear ACE2 importin machinery and displaying minimal toxicity in animal safety studies. (A) The electrophoresis mobility shift assay was carried out to confirm the interaction between IMPα and ACE2 via the C-terminal domain. ACE2 C-terminal domain is FITC labeled. Left panel is Coomassie stained, right panel is visualized by UV. (B) Microscale thermophoresis and fluorescence polarization was used to assess the P604 ACE2 peptide inhibition of binding between importin-a and ACE2. Each experiment was carried out with an n=3, with the KD shown representing the mean and standard deviation. (C) DUOLINK® proximity ligation assay measurements of protein interactions for ACE2^{unmod} and IMPα1 were performed on permeabilized H1299 cells treated with vehicle control or increase doses of P604 ACE2 peptide inhibitor (3.125 mM to 150 mM). The DUOLINK assay produces a single bright dot per interaction within the cell. Representative images are shown for vehicle and lowest dosage (3.125 mM) of P604 ACE2 peptide inhibitor. Graphs of the PLA signal intensity of the DUOLINK® assay is shown for overall average dot intensity (single DUOLINK dot) for each cell, n>20 cells counted. One-Way ANOVA Kruskal Willis was used to calculate differences: ****<0.0001

FIG. 13 provides graphical representation showing P604 ACE2 peptide inhibitor treatment inhibits viral replication and protects against early lung inflammation associated with SARS-Cov-2 infection in SARS-COV2 Syrian golden hamster pre-clinical animal model. (A) qRT-PCR analysis to detect replicates of SARS-CoV-2 RNA in infected lungs from golden Syrian hamsters treated as described above (A). RNA yield is presented as log 10 TCID₅₀ eq/mL. (B) Viral load % is presented relative to Vehicle. Data represent mean±SEM, n=7-8/group. Tukey's post test, ****p<0.0001 denotes significant differences. (C) TCID₅₀ assay to measure infectious viral titers in infected lungs. Data represent mean±SEM, n=5/group. Tukey's post test, ** p<0.01, ***p<0.001 denotes significant differences. (D) Depicts example images of stained Lung FFPE sections of a golden Syrian hamster SARS-CoV-2 infection model that has been infected with SARS-CoV-2 and treated with either vehicle control, NACE2 IP or NACE2i IV with a 20 mM scale bar in yellow. Lung tissue FFPE was processed as described in the methods and stained for SARS-CoV-2 Spike protein. (E) Dot graphs depict the analysis and imaging was carried out using ASI Digital analysis of captured images (n>1000 cells analyzed). Data is plotted with mean±SEM and represents population dynamics of SARS-CoV-2 Spike positive cells and the fluorescent intensity of the SARS-CoV-2 Spike protein. One-Way ANOVA Kruskal-Wallis was used to calculate differences: NS=non-significant, ***=0.0005, ****<0.0001.

FIG. 14 provides a graphical representation showing that aadministration of P604 ACE2 peptide inhibitor reduces SARS-Cov-2 viral infection and inflammation in the lungs of golden Syrian hamsters. (A) Hematoxylin and eosin (H&E) stained tissue sections of lungs from vehicle and P604 ACE2 peptide inhibitor treated animals. Left panel: bronchiolitis with degeneration, necrosis and exfoliation of epithelial cells accompanied by transmural leukocyte infiltration (widespread apoptosis). Middle panel: vasculitis characterized by margination and transmural migration of heterophils and monocytes accompanied by endothelial cell and smooth muscle cell damage (arrow). Also seen in this panel is mild alveolar and interstitial leukocyte accumulation (star). Right panel: No inflammation observed in the vessels or bronchioles. No vascular changes including no infiltration of monocytes or heterotrophs, and no accumulation of alveolar macrophages. Scale bar=200 pm. (B) Pathology scores of infected lungs from H&E stained tissue sections. Note: An additional score of zero was recorded for pneumocyte hyperplasia for all samples. Data represent mean, n =7-8/group. Tukey's post test, *p<0.05 denotes significant differences.

FIG. 15 provides a graphical representation showing that P604 ACE2 peptide inhibitor induces anti-viral signature/effector signature and abrogates nuclear ACE2. (A) Depicts example images of stained lung FFPE sections of a golden Syrian hamster SARS-CoV-2 infection model that has been infected with SARS-CoV-2 and treated with either vehicle control, P604 ACE2 peptide inhibitor IP or P604 ACE2 peptide inhibitor IV. Lung tissue FFPE was processed as described in the methods and stained for perforin and CD3. Analysis and imaging was carried out using ASI Digital analysis of captured images (n>1000 cells analyzed). Data is plotted with mean±SEM and represents population dynamics of CD3 and Perforin positive cells. One-Way ANOVA Kruskal-Wallis was used to calculate differences: NS=non-significant, **=0.0047, ***=0.0002.

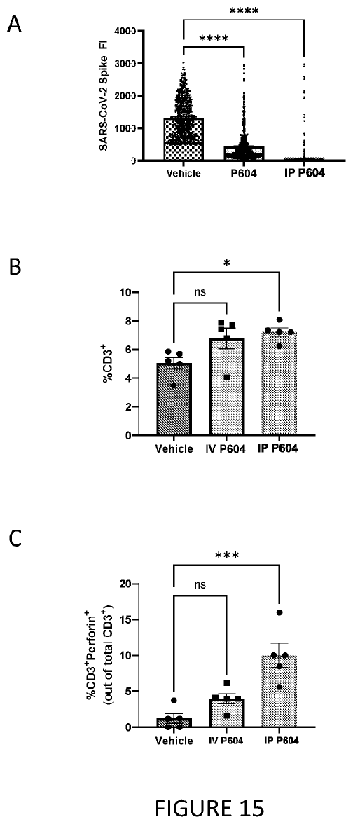

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a cell” means one cell or more than one cell.

The term “about” as used herein refers to the usual error range for the respective value readily known to the skilled person in this technical field. Reference to “about” a value or parameter herein includes (and describes) embodiments that are directed to that value or parameter per se.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 cm, preferably from within about 0.5 to about 5 cm. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogues thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

The term antagonist” or “inhibitor” refers to a substance that prevents, blocks, inhibits, neutralizes, or reduces a biological activity or effect of another molecule, such as a receptor.

As use herein, the term “binds”, “specifically binds to” or is “specific for” refers to measurable and reproducible interactions such as binding between a target and a binding molecule, which is determinative of the presence of the target in the presence of a heterogeneous population of molecules including biological molecules. For example, binding molecule that binds to or specifically binds to a target (which can be an epitope) is a molecule that binds this target with greater affinity, avidity, more readily, and/or with greater duration than it binds to other targets. In one embodiment, the extent of binding of a binding molecule to an unrelated target is less than about 10% of the binding of the molecule to the target as measured, e.g., by a radioimmunoassay (RIA). In certain embodiments, a binding molecule that specifically binds to a target has a dissociation constant (Kd) of ≤1 μM, :≤100 nM, ≤10 nM, ≤1 nM, or ≤0.1 nM. In certain embodiments, a binding molecule specifically binds to a region on a protein that is conserved among the protein from different species. In another embodiment, specific binding can include, but does not require exclusive binding.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “corresponds to” or “corresponding to” is meant an amino acid sequence that displays substantial sequence similarity or identity to a reference amino acid sequence. In general, the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioural symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of an infection, an effective amount of the drug may have the effect in reducing pathogen (bacterium, virus, etc.) titres in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The term “infection” refers to invasion of body tissues by disease-causing microorganisms, their multiplication and the reaction of body tissues to these microorganisms and the toxins they produce. “Infection” includes but are not limited to infections by viruses, prions, bacteria, viroids, parasites, protozoans and fungi. In the context of the present invention, however, “infection” generally refers to virus infection of the family Coronavitidae (e.g., coronaviruses).

As used herein, “instructional material” includes a publication, a recording, a diagram, or any other medium of expression which can be used to communicate the usefulness of the compositions and methods of the invention. The instructional material of the kit of the invention may, for example, be affixed to a container which contains the therapeutic or diagnostic agents of the invention or be shipped together with a container which contains the therapeutic or diagnostic agents of the invention.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (e.g., snakes, frogs, lizards etc.), and fish. A preferred subject is a human in need of a treatment for a SARS-CoV infection, including an SARS-CoV-2 infection. However, it will be understood that the aforementioned terms do not imply that symptoms are present.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

As used herein, the terms “prevent”, “prevented”, or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

The term “sequence identity” as used herein refers to the extent that sequences are identical on a nucleotide-by-nucleotide basis or an amino acid-by-amino acid basis over a window of comparison. Thus, a ““percentage of sequence identity” is calculated by comparing two optimally aligned sequences over the window of comparison, determining the number of positions at which the identical nucleic acid base (e.g., A, T, C, G, 1) or the identical amino acid residue (e.g., Ala, Pro, Ser, Thr, Gly, Val, Leu, lie, Phe, Tyr, Trp, Lys, Arg, His, Asp, Glu, Asn, Gln, Cys, and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software.

As used herein a “small molecule” refers to a compound that has a molecular weight of less than 3 kilodalton (kDa), and typically less than 1.5 kDa, and more preferably less than about 1 kDa. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kDa, less than 1.5 kDa, or even less than about 1 kDa.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridisable sequence, the higher the relative temperature which can pbe used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 15 mM sodium chloride/1.5 mM sodium citrate/0.1% sodium dodecyl sulphate at 50° C.; (2) employ during hybridization a denaturing agent, such as formamide, for example, 50% (v/v) formamide with 0.1% bovine serum albumin/0.1% Fico11/0.1′)/0 polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 pg/mL), 0.1% SDS, and 10% dextran sulphate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein, underscoring or italicizing the name of a gene shall indicate the gene, in contrast to its protein product, which is indicated by the name of the gene in the absence of any underscoring or italicizing. For example, “ACE2” shall mean the ACE2 gene, whereas “ACE2” shall indicate the protein product or products generated from transcription and translation and/or alternative splicing of the ACE2 gene.

Each embodiment described herein is to be applied mutatis mutandis to each and every embodiment unless specifically stated otherwise.

### 2. Proteinaceous Molecules

The present invention is based in part on the determination that the SARS-CoV virus utilises host machinery in order to obtain entry into a host cell, and that these essential host machinery are regulated at the post-translational level and the transcriptional level by methylation and ubiquitination. Without wishing to be bound by any theory or mode of operation, it is proposed that the post-translational methylation/demethylation plays a critical role in at least two levels: (1) regulation of the ACE2 protein interaction with the nuclear transporter, importin-α (IMPα) protein and therefore, nuclear translocation; and (2) regulation of ubiquitination of the ACE2 protein, which signals for protein for proteasomal degradation.

Based on this observation, the present inventors propose that the administration of ACE2 peptides which include a sequence that corresponds to one or more methylation/demethylation sites of the wild-type ACE2 protein will result in reduced ability for SARS-CoV to enter into a host cell, thus providing a novel treatment for coronavirus infections.

Alternatively, or in addition, the ACE2 peptides which include an amino acid sequence that corresponds to the nuclear localisation motif of the wild-type ACE2 protein, which result in inhibition of the interaction between ACE2 and IMPα.

In accordance with the present invention, methods and compositions are provided that take advantage of these ACE2 peptides to reduce or abrogate the transcription of integral cellular machinery required for a coronavirus entry into a cell, as well as to attenuate the signalling of ACE2 protein to the proteasome. In some embodiments, the ACE2 peptide is used in combination with an additional antiviral agent. The methods and compositions of the present invention are thus particularly useful in the treatment or prophylaxis of a coronavirus infection (e.g., a SARS-CoV-2 infection), as described hereafter.

### 2.1 ACE2 Peptides

The present invention is based in part of the determination that the C-terminal tail region of the ACE2 protein plays a pivotal role in its nuclear translocation from the cell surface. The present inventors have also determined that when proteinaceous molecules (e.g., peptides and/or polypeptides) comprising an amino acid sequence that corresponds to the ACE2 protein C-terminal tail region sequence are administered to a subject, these molecules are surprisingly effective as a treatment (including preventative treatment) of a SARS-CoV infection. This activity results, at least in part, from a number of functional capabilities of the ACE2 peptides, including but not limited to: (1) inhibiting the nuclear translocation of the host cell ACE2 protein; (2) inhibiting the ubiquitination of the host cell ACE2 protein; (3) preventing an interaction between an ACE2 peptide or polypeptide and/or an IMPα polypeptide.

In some embodiments, the ACE2 peptide comprises an amino acid sequence that corresponds to at least a portion of the wild-type human ACE2 protein. In some embodiments of this type, the wild-type human ACE2 protein amino acid sequence is that deposited under UniProt Accession No. Q9BYF1, as set forth below:

[SEQ ID NO: 1]
MSSSSWLLLSLVAVTAAQSTIEEQAKTFLDKFNHEAEDLFYQSSLASWNYNTNITEENVQNM
NNAGDKWSAFLKEQSTLAQMYPLQEIQNLTVKLQLQALQQNGSSVLSEDKSKRLNTILNTMS
TIYSTGKVCNPDNPQECLLLEPGLNEIMANSLDYNERLWAWESWRSEVGKQLRPLYEEYVV
LKNEMARANHYEDYGDYWRGDYEVNGVDGYDYSRGQLIEDVEHTFEEIKPLYEHLHAYVRA
KLMNAYPSYISPIGCLPAHLLGDMWGRFWTNLYSLTVPFGQKPNIDVTDAMVDQAWDAQRIF
KEAEKFFVSVGLPNMTQGFWENSMLTDPGNVQKAVCHPTAWDLGKGDFRILMCTKVTMDD
FLTAHHEMGHIQYDMAYAAQPFLLRNGANEGFHEAVGEIMSLSAATPKHLKSIGLLSPDFQE
DNETEINFLLKQALTIVGTLPFTYMLEKWRWMVFKGEIPKDQWMKKWWEMKREIVGVVEPV
PHDETYCDPASLFHVSNDYSFIRYYTRTLYQFQFQEALCQAAKHEGPLHKCDISNSTEAGQK
LFNMLRLGKSEPWTLALENVVGAKNMNVRPLLNYFEPLFTWLKDQNKNSFVGWSTDWSPY
ADQSIKVRISLKSALGDKAYEWNDNEMYLFRS
SVAYAMRQYFLKVKNQMILFGEEDVRVANLKPRISFNFFVTAPKNVSDIIPRTEVEKAIRMSRS
RINDAFRLNDNSLEFLGIQPTLGPPNQPPVSIWLIVFGVVMGVIVVGIVILIFTGIRDRKKKNKAR
SGENPYASIDISKGENNPGFQNTDDVQTSF.

In some embodiments, the ACE2 peptide comprises, consists, or consists essentially of an amino acid sequence that corresponds to the C-terminal tail region (i.e., residues 763-805) of the full-length human ACE2 protein sequence (as set forth in SEQ ID NO: 1), or a fragment thereof.

In some embodiments, the ACE2 peptide includes one or more lysine methylation site(s). For example, lysine residues K26, K353, K769, K770, and K771 of the full-length human ACE2 protein sequence (as set forth above, and SEQ ID NO: 1), are identified as methylation residues, which are shown herein as being LSD-1-mediated methylated/demethylated resides. Accordingly, in some embodiments the present invention provides a proteinaceous molecule that comprises an ACE2 peptide comprising one or more methylation site(s) corresponding to K26, K353, K769, K770, and K771 of the full-length wild-type human ACE2 protein. In some preferred embodiments, the proteinaceous molecule comprises an ACE2 peptides comprising one, two or all of the methylation sites corresponding to residues K769, K770, and K771 of the full-length ACE2 protein. In some of the same embodiments and some other embodiments, the ACE2 peptide also comprises an amino acid residue corresponding to residue K773 of the wild-type human ACE2 protein, which may be a further methylation site.

In some embodiments, at least one of the amino acids corresponding to K769, K770, K771, and K773 of the full-length ACE2 protein is methylated. In some embodiments, at least two of the amino acids corresponding to K769, K770, K771, and K773 of the full-length ACE2 protein is methylated. In some embodiments, at least three of the amino acids corresponding to K769, K770, K771, and K773 of the full-length ACE2 protein are methylated. In some embodiments, each of the amino acids corresponding to K769, K770, K771, and K773 of the full-length ACE2 protein are methylated. In some preferred embodiments, amino acids corresponding to residues K769, K770, and K771 are all methylated.

In some embodiments, at least one of the amino acids corresponding to K769, K770, K771, and K773 of the full-length ACE2 protein is acetylated. In some embodiments, at least two of the amino acids corresponding to K769, K770, K771, and K773 of the full-length ACE2 protein is acetylated. In some embodiments, at least three of the amino acids corresponding to K769, K770, K771, and K773 of the full-length ACE2 protein are acetylated. In some embodiments, each of the amino acids corresponding to K769, K770, K771, and K773 of the full-length ACE2 protein are acetylated. In some preferred embodiments, amino acids corresponding to residues K769, K770, and K771 are all acetylated.

In some of the same embodiments and some alternative embodiments, the ACE2 peptides comprise a residue that corresponds to a ubiquitination site of the wild-type human ACE2 protein. In this regard, protein degradation is well known to be regulated by ubiquitination, and protein methylation has previously been reported as a precursor for protein ubiquitination. Accordingly, in one aspect, preventing or reducing demethylation of host ACE2 protein serves as a mechanism to increase ubiquitination of the protein, and thus stimulate degradation of the protein. Such regulation has previously been observed, for example, with respect to DNMT1 key epigenetic enzyme stability by LSD-1 (see, Yang, Epigenetics). Accordingly, in some embodiments the ACE2 peptide may also comprise an amino acid residue that corresponds to amino acid residue K788 of the full length wild-type human ACE2 protein. By way of an illustrative example, in some embodiments the ACE2 polypeptide may comprise, consist, or consist essentially of the amino acid sequence selected from:

[SEQ ID NO: 19]
DISKGENNPGFQNTDDVQTSF;
[SEQ ID NO: 20]
ASIDISKGENNPGFQNTDD;
or
[SEQ ID NO: 21])
VQTSFDISKGENNPGFQNTDDVQTSF.

In some of the same embodiments and some other embodiments, the proteinaceous molecules prevent or otherwise reduce the binding of an ACE2 polypeptide to an importin-a (IMPa) polypeptide. Suitably, the proteinaceous molecules of this type may comprise, consist or consist essentially of any of the ACE2 peptides as described above. By way of an illustrative example, the ACE2 peptide may comprise the amino acid sequence TGIRDRKKKNKARS [SEQ ID NO: 3].

In some alternative embodiments, the ACE2 peptide may comprise, consist, or consist essentially of an amino acid sequence corresponding to an IMP-a binding region of the wild-type human ACE2 protein (e.g., residues 774 to 787 of the sequence set forth in SEQ ID NO: 1). In some embodiments of this type, the ACE2 peptide comprises, consists, or consists essentially of the amino acid sequence ARSGENPYASIDIS [SEQ ID NO: 18].

Several variants of the native protein amino acid sequence have also been identified.

In some embodiments, the proteinaceous molecules of the invention generally comprise, consist, or consist essentially of an amino acid sequence represented by Formula III:

(Formula III)
[SEQ ID NO: 22]
X₁GIRX₂RX₃X₄X₅X₆X₇AX₈S

X₁ is selected from any small or polar amino acid (preferably, a T or S amino acid), or modified forms thereof;
X₂ is selected from a D or N amino acid, or modified forms thereof;
X₃, X₄, and X₅ are each independently selected from K and Q amino acids, or modified forms thereof;
X₆ is selected from any polar amino acid (e.g., an N, K, or D amino acid), or a modified form thereof;
X₇ is selected from a K or Q amino acid, or a modified form thereof;
X₈ is selected from an R, G, or S amino acid, or a modified form thereof; and

In some preferred embodiments, the ACE2 peptide comprises, consists, or consists essentially of an amino acid sequence that comprises: TGIRDRKKKNKARS [SEQ ID NO: 3].

Such proteinaceous molecules suitably inhibit or reduce the interaction between an ACE2 protein and IMPα. As such, peptides of this type reduce the nuclear localisation of ACE2 protein. This results in a lower level of nuclear ACE2 protein in a cell.

The present invention provides ACE2 peptides in compositions and methods for preventing or reducing the coronavirus entry into a host cell. The present invention also provides compositions and methods for preventing or reducing the replication of a coronavirus in a cell of a subject.

When included in compositions, the ACE2 peptides are suitably combined with a pharmaceutically acceptable carrier or diluent. The ACE2 peptides of the present invention can be administered by any suitable route including, for example, by injection, by topical or mucosal application, by inhalation, or via the oral route including modified-release modes of administration to treat or prevent a coronavirus infection in a subject.

In some embodiments, the ACE2 peptides are obtained using recombinant DNA techniques or by chemical synthesis. Alternatively, the ACE2 peptides may be obtained (e.g., purified or isolated) from a mammalian cell sample.

The ACE2 peptides of the present invention include peptides or polypeptides which arise as a result of the existence of alternative translational and post-translational events. The ACE2 peptides can be expressed in systems (e.g., cultured cells, which result in substantially the same post-translational modifications present when the ACE2 protein is expressed in a native cell, or in systems which result in the alteration or omission of post-translational modifications (e.g., glycosylation or cleavage) present when expressed in a native cell.

The present invention contemplates full-length ACE2 polypeptides as well as their biologically active fragments. Typically, biologically active fragments of a full-length ACE2 polypeptide may participate in an interaction, for example, an intramolecular or an inter-molecular interaction (e.g., an interaction between an IMPα polypeptide). Such biologically active fragments include peptides comprising amino acid sequences sufficiently similar to or derived from the amino acid sequences of a (putative) full-length ACE2 polypeptide, for example, the amino acid sequences shown in SEQ ID NO: 1. A biologically active fragment of a full-length ACE2 peptide can be a peptide which is, for example, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or more amino acids in length.

### N-Terminal ACE2 Peptides

In other embodiments, the ACE2 peptide inhibitors contain a sequence that corresponds to lysine residue 31 of the wild-type human ACE2 sequence. This lysine residue is an integral methylation/demethylation site of the ACE2 polypeptide, the demethylation of which is necessary for interaction with the viral spike protein. Accordingly, the lysine 31 demethylation motif of ACE2 is important for SARS-CoV-2 replication, and thus peptide inhibitors corresponding to this lysine residue have antiviral activity by significantly reducing spike protein co-localization with ACE2.

Thus, in some embodiments the invention provides proteinaceous molecules comprising a peptide with an amino acid sequence represented by Formula IV:

(Formula IV)
Z₁IEEQAKTFLDKZ₂

wherein:
Z₁ is absent or is selected from at least one of a proteinaceous moiety comprising from about 1 to about 50 amino acid residues, and/or a protecting moiety; and
Z₂ is absent or is selected from at least one of a proteinaceous moiety comprising from about 1 to about 50 amino acid residues [SEQ ID NO: 23].

By way of an illustrative example, Z₁ may be absent, and Z₂ may comprise the amino acid sequence FNHEAEDLFYQSSLASWNYNT [SEQ ID NO: 24]. In some preferred embodiments, the proteinaceous molecule of comprises, consists, or consists essentially of, the amino acid sequence: IEEQAKTFLDKFNHEAEDLFYQSSLASWNYNT [SEQ ID NO: 24].

In an alternative example, Z₁ may comprise the amino acid sequence ST, and may be Z₂ absent. Accordingly, in some preferred embodiments, the proteinaceous molecule may comprise, consist, or consist essentially of the amino acid sequence STIEEQAKTFLDK [SEQ ID NO: 26].

In some embodiments, the peptide comprises, consists, or consists essentially of a peptide sequence according to Formula IV. In some embodiments, the sequence comprises a polypeptide sequence according to Formula IV, with one or more single amino acid substitutions in the IEEQAKTFLDK region. In embodiments of this type, a substitution of the lysine that corresponds to lysine 31 of the wild-type human ACE2 amino acid sequence is not tolerated. Accordingly the one or more substitutions may not occur at the lysine that corresponds to lysine 31 of the wild-type human ACE2 polypeptide sequence.

### Variant ACE2 Peptides.

The present invention also contemplates ACE2 peptides that are variants of wild-type or naturally-occurring ACE2 protein or their fragments. Such “variant” peptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein.

Variant proteins encompassed by the present invention are biologically active, that is, they continue to possess a desired biological activity of the native protein (e.g., binding to an LSD1 polypeptide; or binding to an IMP a polypeptide). Such variants may result from, for example, genetic polymorphism or from human manipulation.

An ACE2 peptide or polypeptide may be altered in various ways including amino acid substitutions, deletions, truncations, and insertions. Methods for such manipulations are generally known in the art. For example, amino acid sequence variants of ACE2 peptides or polypeptides can be prepared by mutations in the DNA. Methods for mutagenesis and nucleotide sequence alterations are well known in the art (see, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of ACE2 peptides or polypeptides. Recursive ensemble mutagenesis (REM), a technique which enhances the frequency of functional mutants in the libraries, can be used in combination with the screening assays to identify ACE2 variants (see, Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Variant ACE2 peptides or polypeptides may contain conservative amino acid substitutions at various locations along their sequence, as compared to a parent (e.g., naturally-occurring or reference) ACE2 amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the a-amino group, as well as the a-carbon. Several amino acid similarity matrices (e.g., PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington DC; and by Gonnet et al., (1992, Science, 256(5062): 14430-1445), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table 2.

TABLE 2
AMINO ACID SUB-CLASSIFICATION

| Sub-classes | Amino Acids |
|---|---|
| Acidic | Aspartic acid, Glutamic acid |
| Basic | Noncyclic: Arginine, lysine; |
| | Cyclic: histidine |
| Charged | Aspartic acid, glutamic acid, arginine, lysine, |
| | histidine |
| Small | Glycine, serine, alanine, threonine, proline |
| Nonpolar/neutral | Alanine, glycine, isoleucine, leucine, methionine, |
| | phenylalanine, proline, tryptophan, valine |
| Polar/neutral | Asparagine, histidine, glutamine, cysteine, |
| | serine, threonine, tyrosine |
| Polar/negative | Aspartic acid, glutamic acid |
| Polar/positive | Lysine, arginine |
| Polar/large | Asparagine, glutamine |
| Polar | Arginine, asparagine, aspartic acid, cysteine, |
| | glutamic acid, glutamine, histidine, lysine, |
| | serine, threonine, tyrosine |
| Hydrophobic | Tyrosine, valine, isoleucine, leucine, methionine, |
| | phenylalanine, tryptophan |
| Aromatic | Tryptophan, tyrosine, phenylalanine |
| Residues that | Glycine and proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have. a major effect on the properties of the resulting variant polypeptide. Whether an amino acid change results in a functional ACE2 peptide polypeptide can readily be determined by assaying its activity. Conservative substitutions are shown in Table 3 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 3
EXEMPLARY AND PREFERRED
AMINO ACID SUBSTITUTIONS

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | See |
| Gln | Asn, His, Lys | Asn |
| Glu | Asp, Lys | Asp |
| Gly | Pro | Pro |
| His | Asn, Gln, Lys, Arg | Arg |
| Ile | Leu, Val, Met, Ala, | Leu |
| | Phe, Norleu | |
| Leu | Norleu, Ile, Val, Met, | Ile |
| | Ala, Phe | |
| Lys | Arg, Gln, Asn | Arg |
| Met | Leu, Ile, Phe | Leu |
| Phe | Leu, Vel, Ile, Ala | Leu |
| Pro | Gly | Gly |
| Ser | Thr | Thr |
| Thr | Ser | Ser |
| Trp | Tyr | Tyr |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm:C. Brown Publishers (1993).

Thus, a predicted non-essential amino acid residue in an ACE2 peptide or polypeptide is typically replaced with another amino acid residue from the same side chain family. Alternatively, mutations can be introduced randomly along all or part of an ACE2 gene coding sequence, such as by saturation mutagenesis, and the resultant mutants can be screened for an activity of the parent polypeptide, as described for example herein, to identify mutants which retain that activity. Following mutagenesis of the coding sequences, the encoded peptide or polypeptide can be expressed recombinantly and its activity determined. A “nonessential” amino acid residue is a residue that can be altered from the wild-type sequence of an embodiment peptide or polypeptide without abolishing or substantially altering one or more of its activities. Suitably, the alteration does not substantially alter one of these activities, for example, the activity is at least 20%, 40%, 60%, 70% or 80% of wild-type. By contrast, an “essential” amino acid residue is a residue that, when altered from the wild-type sequence of a reference ACE2 peptide or polypeptide, results in abolition of an activity of the parent molecule such that less than 20% of the wild-type activity is present. For example, such essential amino acid residues include those that are conserved in ACE2 peptides or polypeptides across different species.

Accordingly, the present invention also contemplates as ACE2 peptides or polypeptides, variants of the naturally-occurring ACE2 polypeptide sequences or their biologically-active fragments, wherein the variants are distinguished from the naturally-occurring sequence by the addition, deletion, or substitution of one or more amino acid residues. In general, variants will display at least about 40%, 45%, 50%, 51%, 52%, 53%, 54%, 55%, 56%, 57%, 58%, 59%, 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% similarity to a parent or reference ACE2 peptide or polypeptide sequence as, for example, set forth in SEQ ID NO: 1, as determined by sequence alignment programs described elsewhere herein using default parameters. Desirably, variants will have at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to a parent ACE2 peptide or polypeptide sequence as, for example, set forth in SEQ ID NO: 1, as determined by sequence alignment programs described elsewhere herein using default parameters. Variants of a wild-type ACE2 polypeptide, which fall within the scope of a variant polypeptide, may differ from the wild-type molecule generally by as much 15, 14, 13, 12, or 11 amino acid residues or suitably by as few as 10, 9, 8, 7, 6, 5 4, 3, 2, or 1 amino acid residue(s). In some embodiments, a variant polypeptide differs from the corresponding sequences in SEQ ID NO: 1 by at least 1 but by less than or equal to 15, 14, 13, 12, 11, 10, 9, 8, 7, 6, 5, 4, 3, or 2 amino acid residues. In other embodiments, it differs from the corresponding sequence in any one of SEQ ID NO: 1 by at least one 1% but less than or equal to 20%, 19%, 18%, 17%, 16%, 15%, 14%, 13%, 12%, 11%, 10%, 9%, 8%, 7%, 6%, 5%, 4%, 3%, or 2% of the residues. If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution, as discussed in more detail below.

The ACE2 peptides of the present invention also encompass ACE2 peptide or polypeptides comprising amino acids with modified side chains, incorporation of unnatural amino acid residues and/or their derivatives during peptide, polypeptide or protein synthesis and the use of cross-linkers and other methods which impose conformational constraints on the peptides, portions, and variants of the invention. Examples of side chain modifications include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBRt; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivatization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in Table 4.

TABLE 4
NON-CONVENTIONAL AMINO ACIDS

| | |
|---|---|
| α-aminobutyric acid | L-N-methylalanine |
| α-amino-α-methylbutyrate | L-N-methylarginine |
| Aminocyclopropane-carboxylate | L-N-methylasparagine |
| Aminoisobutyric acid | L-N-methylaspartic acid |
| Aminonorbornyl-carboxylate | L-N-methylcysteine |
| Cyclohexylalanine | L-N-methylglutamine |
| Ayclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-β-aminobutyrate |
| D-α-methylalanine | α-methylcyclohexylalanine |
| D-α-methylasparagine | D-α-methyl-α-napthylalanine |
| D-α-methylaspartate | D-α-methylpenicillamine |
| D-α-methylcysteine | N-(4-aminobutyl)glycine |
| D-α-methylhistidine | N-(2-aminoethyl)glycine |
| D-α-methylisoleucine | N-(3-aminopropyl)glycine |
| D-α-methylleucine | N-amino-α-methylbutyrate |
| D-α-methyllysine | α-napthylalanine |
| D-α-methylmethionine | N-benzylglycine |
| D-α-methylornithine | N-(2-carbamylediyl)glycine |
| D-α-methylpheylalanine | N-(caramylmethyl)glycine |
| D-α-methylproline | N-(2-carboxyethyl)glycine |
| D-α-methylserine | N-(carboxymethyl)glycine |
| D-α-methylthreonine | N-cyclobutylglycine |
| D-α-methyltryptophan | N-cycloheptylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-2,2-diphenylethyl | N-(N-(3,3-diphenylpropyl |
| carbamylmethyl)glycine | carbnamylmethyl)glycine |
| 1-carboxy-1-1(2,2-diphenyl- | |
| ethyl amino)cyclopropane | |

The ACE2 peptides of the present invention also include those that are encoded by polynucleotides that hybridize under stringency conditions as defined herein, especially medium or high stringency conditions, to ACE2-encoding polynucleotide sequences, or the non-coding strand thereof, as described below. An illustrative ACE2 polynucleotide sequence is set forth below:

[SEQ ID NO: 2]
AGTCTAGGGAAAGTCATTCAGTGGATGTGATCTTGGCTCACAGGGGACGATGTCAAGC
TCTTCCTGGCTCCTTCTCAGCCTTGTTGCTGTAACTGCTGCTCAGTCCACCATTGAGGA
ACAGGCCAAGACATTTTTGGACAAGTTTAACCACGAAGCCGAAGACCTGTTCTATCAAA
GTTCACTTGCTTCTTGGAATTATAACACCAATATTACTGAAGAGAATGTCCAAAACATGA
ATAATGCTGGGGACAAATGGTCTGCCTTTTTAAAGGAACAGTCCACACTTGCCCAAATG
TATCCACTACAAGAAATTCAGAATCTCACAGTCAAGCTTCAGCTGCAGGCTCTTCAGCA
AAATGGGTCTTCAGTGCTCTCAGAAGACAAGAGCAAACGGTTGAACACAATTCTAAATA
CAATGAGCACCATCTACAGTACTGGAAAAGTTTGTAACCCAGATAATCCACAAGAATGC
TTATTACTTGAACCAGGTTTGAATGAAATAATGGCAAACAGTTTAGACTACAATGAGAGG
CTCTGGGCTTGGGAAAGCTGGAGATCTGAGGTCGGCAAGCAGCTGAGGCCATTATATG
AAGAGTATGTGGTCTTGAAAAATGAGATGGCAAGAGCAAATCATTATGAGGACTATGGG
GATTATTGGAGAGGAGACTATGAAGTAAATGGGGTAGATGGCTATGACTACAGCCGCG
GCCAGTTGATTGAAGATGTGGAACATACCTTTGAAGAGATTAAACCATTATATGAACATC
TTCATGCCTATGTGAGGGCAAAGTTGATGAATGCCTATCCTTCCTATATCAGTCCAATTG
GATGCCTCCCTGCTCATTTGCTTGGTGATATGTGGGGTAGATTTTGGACAAATCTGTAC
TCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTT
GGTCTTCCTAATATGACTCAAGGATTCTGGGAAAATTCCATGCTAACGGACCCAGGAAA
TGTTCAGAAAGCAGTCTGCCATCCCACAGCTTGGGACCTGGGGAAGGGCGACTTCAG
GATCCTTATGTGCACAAAGGTGACAATGGACGACTTCCTGACAGCTCATCATGAGATGG
GGCATATCCAGTATGATATGGCATATGCTGCACAACCTTTTCTGCTAAGAAATGGAGCT
AATGAAGGATTCCATGAAGCTGTTGGGGAAATCATGTCACTTTCTGCAGCCACACCTAA
GCATTTAAAATCCATTGGTCTTCTGTCACCCGATTTTCAAGAAGACAATGAAACAGAAAT
AAACTTCCTGCTCAAACAAGCACTCACGATTGTTGGGACTCTGCCATTTACTTACATGTT
AGAGAAGTGGAGGTGGATGGTCTTTAAAGGGGAAATTCCCAAAGACCAGTGGATGAAA
AAGTGGTGGGAGATGAAGCGAGAGATAGTTGGGGTGGTGGAACCTGTGCCCCATGAT
GAAACATACTGTGACCCCGCATCTCTGTTCCATGTTTCTAATGATTACTCATTCATTCGA
TATTACACAAGGACCCTTTACCAATTCCAGTTTCAAGAAGCACTTTGTCAAGCAGCTAAA
CATGAAGGCCCTCTGCACAAATGTGACATCTCAAACTCTACAGAAGCTGGACAGAAACT
GTTCAATATGCTGAGGCTTGGAAAATCAGAACCCTGGACCCTAGCATTGGAAAATGTTG
TAGGAGCAAAGAACATGAATGTAAGGCCACTGCTCAACTACTTTGAGCCCTTATTTACC
TGGCTGAAAGACCAGAACAAGAATTCTTTTGTGGGATGGAGTACCGACTGGAGTCCAT
ATGCAGACCAAAGCATCAAAGTGAGGATAAGCCTAAAATCAGCTCTTGGAGATAAAGCA
TATGAATGGAACGACAATGAAATGTACCTGTTCCGATCATCTGTTGCATATGCTATGAG
GCAGTACTTTTTAAAAGTAAAAAATCAGATGATTCTTTTTGGGGAGGAGGATGTGCGAG
TGGCTAATTTGAAACCAAGAATCTCCTTTAATTTCTTTGTCACTGCACCTAAAAATGTGT
CTGATATCATTCCTAGAACTGAAGTTGAAAAGGCCATCAGGATGTCCCGGAGCCGTATC
AATGATGCTTTCCGTCTGAATGACAACAGCCTAGAGTTTCTGGGGATACAGCCAACACT
TGGACCTCCTAACCAGCCCCCTGTTTCCATATGGCTGATTGTTTTTGGAGTTGTGATGG
GAGTGATAGTGGTTGGCATTGTCATCCTGATCTTCACTGGGATCAGAGATCGGAAGAA
GAAAAATAAAGCAAGAAGTGGAGAAAATCCTTATGCCTCCATCGATATTAGCAAAGGAG
AAAATAATCCAGGATTCCAAAACACTGATGATGTTCAGACCTCCTTTTAGAAAAATCTAT
GTTTTTCCTCTTGAGGTGATTTTGTTGTATGTAAATGTTAATTTCATGGTATAGAAAATAT
AAGATGATAAAGATATCATTAAATGTCAAAACTATGACTCTGTTCAGAAAAAAAATTGTC
CAAAGACAACATGGCCAAGGAGAGAGCATCTTCATTGACATTGCTTTCAGTATTTATTTC
TGTCTCTGGATTTGACTTCTGTTCTGTTTCTTAATAAGGATTTTGTATTAGAGTATATTAG
GGAAAGTGTGTATTTGGTCTCACAGGCTGTTCAGGGATAATCTAAATGTAAATGTCTGTT
GAATTTCTGAAGTTGAAAACAAGGATATATCATTGGAGCAAGTGTTGGATCTTGTATGG
AATATGGATGGATCACTTGTAAGGACAGTGCCTGGGAACTGGTGTAGCTGCAAGGATT
GAGAATGGCATGCATTAGCTCACTTTCATTTAATCCATTGTCAAGGATGACATGCTTTCT
TCACAGTAACTCAGTTCAAGTACTATGGTGATTTGCCTACAGTGATGTTTGGAATCGATC
ATGCTTTCTTCAAGGTGACAGGTCTAAAGAGAGAAGAATCCAGGGAACAGGTAGAGGA
CATTGCTTTTTCACTTCCAAGGTGCTTGATCAACATCTCCCTGACAACACAAAACTAGAG
CCAGGGGCCTCCGTGAACTCCCAGAGCATGCCTGATAGAAACTCATTTCTACTGTTCTC
TAACTGTGGAGTGAATGGAAATTCCAACTGTATGTTCACCCTCTGAAGTGGGTACCCAG
TCTCTTAAATCTTTTGTATTTGCTCACAGTGTTTGAGCAGTGCTGAGCACAAAGCAGACA
CTCAATAAATGCTAGATTTACACACTC.

In some embodiments, calculations of sequence similarity or sequence identity between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percent identity between the two sequences is a function of the number of identical amino acid residues shared by the sequences at individual positions, taking into account the number of gaps, and the length of each gap, which need to be introduced for optimal alignment of the two sequences. By contrast, the percent similarity between the two sequences is a function of the number of identical and similar amino acid residues shared by the sequences at individual positions, taking into account the number of gaps, and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wunsch, (1970, J. Mol. Biol. 48: 444-453) algorithm which has been incorporated into the GAP program in the GCG software package (www.qcq.com) using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (www.qcq.com), using a NWSgapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percent identity or similarity between amino acid or nucleotide sequences can be determined using the algorithm of E. Meyers and W. Miller (1989, Cabios, 4: 1 1-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to 53010 protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Variants of a reference ACE2 peptide or polypeptide can be identified by screening combinatorial libraries of mutants, e.g., truncation mutants, of an ACE2 peptide or polypeptide. Libraries or fragments e.g., N-terminal, C-terminal, or internal fragments, of an ACE2 coding sequence can be used to generate a variegated population of fragments for screening and subsequent selection of variants of a reference ACE2.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of ACE2 peptides or polypeptides.

The ACE2 peptides and polypeptides of the present invention may be prepared by any suitable procedure known to those of skill in the art. For example, the ACE2 peptides or polypeptides may be produced by any convenient method such as by purifying the peptides or polypeptides from naturally-occurring reservoir. Methods of purification include size exclusion, affinity or ion exchange chromatography/separation. The identity and purity of derived ACE2 peptides is determined for example by SDS-polyacrylamide gel electrophoresis (SDS-PAGE) or chromatographically such as by high performance liquid chromatography (HPLC). Alternatively, the ACE2 peptides or polypeptides may be synthesized by chemical synthesis, e.g., using solution synthesis or solid phase synthesis as described, for example, in Chapter 9 of Atherton and Shephard (supra) and in Roberge et al., (1995, Science, 269: 202).

In some embodiments, the ACE2 peptides or polypeptides are prepared by recombinant techniques. For example, the ACE2 peptides or polypeptides of the invention may be prepared by a procedure including the steps of: (a) preparing a construct comprising a polynucleotide sequence that encodes an ACE2 peptide or polypeptide and that is operably linked to a regulatory element; (b) introducing the construct into a host cell; (c) culturing the host cell to express the polynucleotide sequence to thereby produce the encoded ACE2 peptide or polypeptide; and (d) isolating the ACE2 peptide or polypeptide from the host cell. In illustrative examples, the nucleotide sequence encodes at least a biologically active portion of the sequences set forth in SEQ ID NO: 3, or a variant thereof. Recombinant ACE2 peptides or polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6.

In some embodiments, the ACE2 peptides are homologues or orthologues to the wild-type human ACE2 amino acid sequences. Although a high degree of sequence identity exists between orthologues, there is some tolerance for variant amino acid residues at several residues of the C-terminal tail. For example, the ACE2 peptide may comprise any one of the following sequences: an ACE2 peptide from human (TGIRDRKKKNKARSGENPYASIDISKGENNPGFQNTDDVQTSF) or a fragment thereof [SEQ ID NO: 12]; an ACE2 peptide from Myotis lucifugus (TGIRDRKKKKQAGNEENPYSSVNLSKGENNPGFQNGDDVQTSF) or a fragment thereof [SEQ ID NO: 25]; an ACE2 peptide from Felis catus (SGIRNRRKNNQARSEENPYASVDLSKGENNPGFQHADDVQTSF) or a fragment thereof [SEQ ID NO: 27]; an ACE2 peptide from Canis lupus familiaris (SGIRNRRKNDQARGEENPYASVDLSKGENNPGFQNVDDAQTSF) or a fragment thereof [SEQ ID NO: 28]; an ACE2 peptide from Camelus ferus (TGIRDRRKKKQASTEENPYGSVDLSKGENNSGFQNGDDVQTSF) or a fragment thereof [SEQ ID NO: 29]; an ACE2 peptide from Macaca fascicularis (TGIRDRKKKNQARSEENPYASIDINKGENNPGFQNTDDVQTSF) [SEQ ID NO: 30].

Even higher sequence identity exists across the region corresponding to the nuclear translocation site (e.g., corresponding to resides 767-776 of the human ACE2 protein sequence set forth in SEQ ID NO: 1). For example, in some embodiments the ACE2 peptide comprises an amino acid sequence that corresponds to the ACE2 protein NLS amino acid sequence from human ACE2 (DRKKKNKARS) [SEQ ID NO: 31]; the NLS peptide from Myotis lucifugusACE2 (DRKKKKQAGN) [SEQ ID NO: 32]; Felis catus ACE2 (NRRKNNQARS) [SEQ ID NO: 33]; Canis lupus familiaris ACE2 (NRRKNDQARG) [SEQ ID NO: 34]; Camelus ferus ACE2 (DRRKKKQAST) [SEQ ID NO: 35]; or Macaca fascicularis ACE2 (DRKKKNQARS) [SEQ ID NO: 36].

Exemplary nucleotide sequences that encode the ACE2 peptides and polypeptides of the invention encompass full-length ACE2 genes as well as portions of the full-length or substantially full-length nucleotide sequences of the ACE2 genes or their transcripts or ACE2 copies of these transcripts. Portions of an ACE2 nucleotide sequence may encode polypeptide portions or segments that retain a biological activity of the native polypeptide (e.g., nuclear translocation). A portion of an ACE2 nucleotide sequence that encodes a biologically active fragment of an ACE2 polypeptide may encode at least about 7, 8, 9, 10, 11, 12, 13, 14, 15, 16, 17, 18, 19, 20, 21, 22, 23, 24, 25, 26, 27, 28, 29, 30, or more contiguous amino acid residues, or almost up to the total number of amino acids present in a full-length ACE2 polypeptide.

The invention also contemplates variants of the ACE2 nucleotide sequences. Nucleic acid variants can be naturally-occurring, such as allelic variants (same locus), homologues (different locus), and orthologues (different organism) or can be non-naturally occurring. Naturally occurring nucleic acid variants (also referred to herein as polynucleotide variants) such as these can be identified with the use of well-known molecular biology techniques, as, for example, with polymerase chain reaction (PCR) and hybridization techniques as known in the art. Non-naturally occurring polynucleotide variants can be made by mutagenesis techniques, including those applied to polynucleotides, cells, or organisms. The variants can contain nucleotide substitutions, deletions, inversions and insertions.

Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference ACE2 peptide or polypeptide. Variant nucleotide sequences also include synthetically derived nucleotide sequences, such as those generated, for example, by using site-directed mutagenesis but which still encode an ACE2 peptide or polypeptide. Generally, variants of a particular ACE2 nucleotide sequence will have at least about 40%, 45%, 50%, 51%, 52%, 53%, 54%, 55%, 56%, 57%, 58%, 59%, 60%, 61%, 62, %, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, 99% or more sequence identity to that particular nucleotide sequence as determined by sequence alignment programs described elsewhere herein using default parameters. In some embodiments, the ACE2 nucleotide sequence displays at least about 40%, 45%, 50%, 51%, 52%, 53%, 54%, 55%, 56%, 57%, 58%, 59%, 60%, 61%, 62, %, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, 99% or more sequence identity to the nucleotide sequence of SEQ ID NO: 2, or its complement.

ACE2 nucleotide sequences can be used to isolate corresponding sequences and alleles from other organisms, particularly other virus hosts. Methods are readily available in the art for the hybridization of nucleic acid sequences. Coding sequences from other organisms may be isolated according to well-known techniques based on their sequence identity with the coding sequences set forth herein. In these techniques all or part of the known coding sequence is used as a probe which selectively hybridizes to other ACE2-coding sequences present in a population of cloned genomic DNA fragments or cDNA fragments (i.e., genomic or cDNA libraries) from a chosen organism (e.g., a mammal). Accordingly, the present invention also contemplates polynucleotides that hybridize to reference ACE2 nucleotide sequences, or to their complements under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ pH 7.2), 5% SDS for washing at room temperature. One embodiment of low stringency conditions includes hybridization in 6×sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, an ACE2 peptide or polypeptide is encoded by a polynucleotide that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the T, or formation of a DNA-DNA hybrid. It is well known in the art that the T, is the melting temperature, or temperature at which two complementary polynucleotide sequences dissociate. Methods for estimating T, are well known in the art (see, Ausubel et al., supra, at page 2.10.8). In general, the T, of a perfectly matched duplex of DNA may be predicted as an approximation by the formula:

$${T}_{m} = 81.5 + 16.6 ( {log}_{10} M ) + 0.41 ( \% G + C ) - 0.63 ( \% formamide ) - ( 600 / length )$$

wherein: M is the concentration of Na+, preferably in the range of 0.01 molar to 0.4 molar; % G+C is the sum of guanosine and cytosine bases as a percentage of the total number of bases, within the range between 30% and 75% G+C; formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% Ficoll, 0.1% polyvinylpyrrolidone and 0.1% BSA), 0.1% SDS and 200 mg/mL denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

The present invention also contemplates the use of ACE2 chimeric or fusion proteins for treating or preventing undesirable or deleterious immune responses. As used herein, an ACE2 “chimeric protein” or “fusion protein” includes an ACE2 peptide or polypeptide linked to a non-ACE2 peptide or polypeptide. A “non-ACE2 peptide or polypeptide” refers to a peptide or polypeptide having an amino acid sequence corresponding to a protein which is different from native ACE2 and which is derived from the same or a different organism. The ACE2 peptide or polypeptide of the fusion protein can correspond to all or a portion e.g., a fragment described herein of an ACE2 polypeptide amino acid sequence. In a specific embodiment, an ACE2 fusion protein includes at least one biologically active portion of an ACE2 polypeptide. The non-ACE2 peptide or polypeptide can be fused to the N-terminus or C-terminus of the ACE2 peptide or polypeptide.

The fusion protein can include a moiety which has a high affinity for a ligand. For example, the fusion protein can be a GST-ACE2 fusion protein in which the ACE2 sequence is fused to the C-terminus of the GST sequence. Such fusion proteins can facilitate the purification of recombinant ACE2 peptide or polypeptide. Alternatively, the fusion protein can be an ACE2 protein containing a heterologous signal sequence at its N-terminus. In certain host cells (e.g., mammalian host cells), expression and/or secretion of ACE2 peptides or polypeptides can be increased through use of a heterologous signal sequence. In some embodiments, fusion proteins may include all or a part of a serum protein, e.g., an IgG constant region, or human serum albumin.

The ACE2 fusion proteins of the invention can be incorporated into pharmaceutical compositions and administered to a subject in vivo. They can also be used to modulate the bioavailability of an ACE2 peptide or polypeptide.

### 3. Therapeutic Compositions

The present inventors have determined that the C-terminal domain of the native ACE2 protein (i.e., corresponding to amino acid residues 763-805 of the native human ACE2 protein as set forth in SEQ ID NO: 1) plays an important role in a number of activities important for SARS-CoV infection. Namely, these activities include: (i) facilitating virus entry (for example, by engaging the SARS-CoV Spike protein); (ii) nuclear translocation (by binding to the nuclear shuttle protein IMPa); and (iii) targeting the ACE2 protein for proteasomal degradation (through ubiquitination by E3 ligase). Importantly, each of these functions is regulated directly or indirectly by the LSD1-mediated methylation/demethylation of the methylation site(s) present on the C-terminal tail region of the ACE2 protein.

Therefore, in accordance with the present invention, prevention of SARS-CoV virus replication can be achieved using at least one ACE2 peptide as described above or elsewhere herein, or a polynucleotide from which one is expressible, and optionally an antiviral agent.

### 3.1 Pharmaceutical Formulations

In accordance with the present invention, bioactive agents selected from an ACE2 peptide or polypeptide; and optionally an antiviral agent are useful in compositions and methods for treating a coronavirus infection, and more particularly, for preventing or reducing coronavirus replication in a host cell. These compositions are useful, therefore, for treating or preventing a coronavirus infection.

Pharmaceutical compositions suitable for use in the present invention include compositions wherein the bioactive agents are contained in an effective amount to achieve their intended purpose. The dose of active compound(s) administered to a patient should be sufficient to achieve a beneficial response in the patient over time such as a reduction in at least one symptom associated with the unwanted or deleterious immune response, which is suitably associated with a condition selected from an allergy, an autoimmune disease and a transplant rejection. The quantity or dose frequency of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the treatment or prophylaxis of the unwanted or deleterious immune response, the practitioner may evaluate inflammation, pro-inflammatory cytokine levels, lymphocyte proliferation, cytolytic T lymphocyte activity and regulatory T lymphocyte function. In any event, those of skill in the art may readily determine suitable dosages of the antagonist and antigen.

Accordingly, the bioactive agents are administered to a subject to be treated in a manner compatible with the dosage formulation, and in an amount that will be prophylactically and/or therapeutically effective. The amount of the composition to be delivered, generally in the range of from 0.01 pg/kg to 100 μg/kg of bioactive molecule (e.g., ACE2 peptide, antiviral agent, etc.) per dose, depends on the subject to be treated. In some embodiments, and dependent on the intended mode of administration, the ACE2 peptide-containing compositions will generally contain about 0.1% to 90%, about 0.5% to 50%, or about 1% to about 25%, by weight ACE2 the remainder being suitable pharmaceutical carriers and/or diluents etc and optionally the antiviral agent. The dosage of the inhibitor can depend on a variety of factors, such as mode of administration, the species of the affected subject, age and/or individual condition. In other embodiments, and dependent on the intended mode of administration, antiviral agent-containing compositions will generally contain about 0.1% to 90%, about 0.5% to 50%, or about 1% to about 25%, by weight of antiviral agent, the remainder being suitable pharmaceutical carriers and/or diluents etc and the ACE2 peptide or polypeptide.

Depending on the specific nature of the infection being treated, the particles may be formulated and administered systemically, locally, or topically. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, transcutaneous, intradermal, intramedullary delivery (e.g., injection), as well as intrathecal, direct intraventricular, intravenous, intraperitoneal, intranasal, or intraocular delivery (e.g., injection). For injection, the bioactive agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the bioactive agents of the present invention can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the particles in water-soluble form. Additionally, suspensions of the bioactive agents may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the bioactive agents with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The bioactive agents of the present invention may be administered over a period of hours, days, weeks, or months, depending on several factors, including the severity of the condition being treated, whether a recurrence of the condition is considered likely, etc. The administration may be constant, e.g., constant infusion over a period of hours, days, weeks, months, etc. Alternatively, the administration may be intermittent, e.g., bioactive agents may be administered once a day over a period of days, once an hour over a period of hours, or any other such schedule as deemed suitable.

The bioactive agents of the present invention may also be administered to the respiratory tract as a nasal or pulmonary inhalation aerosol or solution for a nebulizer, or as a microfine powder for insufflation, alone or in combination with an inert carrier such as lactose, or with other pharmaceutically acceptable excipients.

In other specific particulate embodiments, the route of particle delivery is via the gastrointestinal tract, e.g., orally. Alternatively, the particles can be introduced into organs such as the lung (e.g., by inhalation of powdered microparticles or of a nebulized or aerosolized solution containing the microparticles), where the particles are picked up by the alveolar macrophages, or may be administered intranasally or buccally. Once a phagocytic cell phagocytoses the particle, the ACE2 peptide and optionally the antiviral agent are released into the interior of the cell.

### 3. Methods of Preventing ACE2 Nuclear Translocation and SARS-CoV Host Cell Entry and/or Replication

The present inventors have determined that post-translational modifications play a significant role in the regulation of functional activity of the viral cell entry receptor polypeptides, particularly, the ACE2 protein. For example, a plurality of methylation sites are identified in both (i) the nuclear localisation sequence (NLS) of the ACE2 protein; and (ii) the catalytic domain of the ACE2 protein. Accordingly, administering the ACE2 peptides of the invention reduces the demethylation activity asserted on the host ACE2 protein (e.g., through competitive inhibition of the LSD1 protein), which results in a number of advantageous activities (e.g., allowing ubiquitination of the ACE2 to signal for proteasomal degradation; inhibition/reduction of the ACE2 protein binding to IMPa; and thus, a reduction of the ACE2 protein nuclear translocation, etc). Accordingly, in some embodiments, ACE2 peptides (or proteinaceous molecules that comprise ACE2 peptide sequences) are administered to a subject to prevent or reduce viral replication of the SARS-CoV in the host cell.

Accordingly, in some embodiments, the proteinaceous molecules of the invention prevent ACE2 nuclear translocation by inhibiting or reducing the binding of the ACE2 to IMPa. In some preferred embodiments of this type, the present invention comprises a polypeptide that corresponds to the NLS of ACE2 (i.e., the amino acid sequence set forth in SEQ ID NO: 3).

Alternatively or in addition, the present invention extends to a method of inhibiting the entry of a betacoronavirus into a cell of the host, the method comprising administering to the subject an ACE2 peptide as described above and/or elsewhere herein. Without wishing to be bound by any particular theory or mechanism, by inhibiting the LSD1 demethylation of the host ACE2 protein, the protein is targeted for proteasomal degradation (by subsequent ubiquitination by a E3 ligase) rather than being transported to the nucleus. The nuclear translation of the ACE2 protein is essential for ACE2 to assert its activity in viral replication of the SARS-CoV.

In some particularly important embodiments of the present invention, the coronavirus is a SARS-CoV-2.

### 5. Therapeutic and Prophylactic Uses

In accordance with the present invention, it is proposed that proteinaceous molecules that inhibit LSD1-mediated demethylation of the ACE2 protein (e.g., ACE2 peptides described above and/or elsewhere herein) are useful as actives and/or pharmaceutical compositions for treating or preventing a virus infection (e.g., a SARS-CoV infection). In such embodiments, it is considered that treatment or prevention includes the prevention of incurring a symptom, holding in check such symptoms, or treating existing symptoms associated with the SARS-CoV infection, when administered to an individual in need thereof.

The proteinaceous molecules of the invention that reduce the ACE2 nuclear localisation (e.g., by preventing the interaction between ACE2 and IMPα) when administered to a subject (e.g., a mammal) result in an increased expression of CD3+Perforin+ cells in the lung. Furthermore, administering these proteinaceous molecules to a subject (e.g., a mammal) with a SARS-CoV-2 infection results in a decrease in inflammation. In some embodiments, the decrease in inflammation occurs in the lung of the subject. Preferably the subject is a mammal, and even more preferably, a human.

Any of the ACE2 peptides described above, or elsewhere herein, can be used in the compositions and methods of the present invention, provided that the inhibitor is pharmaceutically active. A “pharmaceutically active” ACE2 peptide is in a form that results in the treatment and/or prevention of a SARS-CoV infection, particularly a SARS-CoV-2 infection, including the prevention of incurring a symptom, holding in check such symptoms, or treating existing symptoms associated with the infection, when administered to an individual in need thereof.

Modes of administration, amounts of ACE2 peptide administered, and ACE2 peptide formulations, for use in the methods of the present invention, are routine and within the skill of practitioners in the art. Whether a SARS-CoV infection, particularly a SARS-CoV-2 infection, has been treated is determined by measuring one or more diagnostic parameters indicative of the course of the disease, compared to a suitable control. In the case of an animal experiment, a “suitable control” is an animal not treated with the ACE2 peptide, or treated with the pharmaceutical composition without the ACE2 peptide. In the case of a human subject, a “suitable control” may be the individual before treatment, or may be a human (e.g., an age-matched or similar control) treated with a placebo. In accordance with the present invention, the treatment of a SARS-CoV infection includes and encompasses without limitation: (1) preventing the uptake of a SARS-CoV virus (e.g., a SARS-CoV-2 virus) into a cell of the host; (2) treating a SARS-CoV infection (e.g., a SARS-CoV-2 infection) in a subject; (3) preventing a SARS-CoV infection (e.g., a SARS-CoV-2 infection) in a subject that has a predisposition to the SARS-CoV infection but has not yet been diagnosed with the SARS-CoV infection and, accordingly, the treatment constitutes prophylactic treatment of the SARS-CoV infection; or (iii) causing regression of a SARS-CoV infection (e.g., a SARS-CoV-2 infection).

The compositions and methods of the present invention are thus suitable for treating an individual who has been diagnosed with a coronavirus infection, who is suspected of having a SARS-CoV infection, who is known to be susceptible and who is considered likely to develop a SARS-CoV infection, or who is considered likely to develop a recurrence of a previously treated SARS-CoV infection. Typically, the coronavirus infection is a SARS-CoV-1 or a SARS-CoV-2 infection. In some preferred embodiments, the coronavirus infection is a SARS-CoV-2 infection.

In some embodiments, and dependent on the intended mode of administration, the ACE2 peptide-containing compositions will generally contain about 0.000001% to 90%, about 0.0001% to 50%, or about 0.01% to about 25%, by weight of ACE2 peptide, the remainder being suitable pharmaceutical carriers or diluents etc. The dosage of the ACE2 peptide can depend on a variety of factors, such as mode of administration, the species of the affected subject, age, sex, weight and general health condition, and can be easily determined by a person of skill in the art using standard protocols. The dosages will also take into consideration the binding affinity of the ACE2 peptide to its target molecule (e.g., IMPα, LSD1, etc), its bioavailability and its in vivo and pharmacokinetic properties. In this regard, precise amounts of the agents for administration can also depend on the judgment of the practitioner. In determining the effective amount of the agents to be administered in the treatment or prevention of a pathogenic infection, the physician or veterinarian may evaluate the progression of the disease or condition over time. In any event, those of skill in the art may readily determine suitable dosages of the LSD1 inhibitor without undue experimentation. The dosage of the actives administered to a patient should be sufficient to effect a beneficial response in the patient over time such as impairment, abrogation or prevention in the uptake of the virus into a cell of the host, and/or in the treatment and/or prevention of a SARS-CoV infection (e.g., a coronavirus infection, for example, a SARS-CoV-2 infection). The dosages may be administered at suitable intervals to ameliorating the symptoms of the hematologic malignancy. Such intervals can be ascertained using routine procedures known to persons of skill in the art and can vary depending on the type of active agent employed and its formulation. For example, the interval may be daily, every other day, weekly, fortnightly, monthly, bimonthly, quarterly, half-yearly or yearly.

Dosage amount and interval may be adjusted individually to provide plasma levels of the active agent, which are sufficient to maintain its inhibitory effects. Usual patient dosages for systemic administration range from 1-2000 mg/day, commonly from 1250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

In accordance with the practice of the present invention, inhibition of LSD (e.g., LSD1 and LSD2) by the ACE2 peptide will result in reduced levels of ACE2 protein on the cell surface, and thus reduced uptake of the virus into cells of the host. This will, in turn, result in fewer virus infected cells. Accordingly, it would be expected that a more effective treatment of the virus infection with an auxiliary cancer therapy or agent would occur. Thus, the present invention further contemplates administering the ACE2 peptide concurrently with at least one antiviral agent. The ACE2 peptide may be used therapeutically after the antiviral agent or may be used before the antiviral agent is administered or together with the antiviral agent. Accordingly, the present invention contemplates combination therapies, which employ an ACE2 peptide and concurrent administration of an antiviral agent, non-limiting examples of which include: broad-spectrum antiviral agents and coronavirus-specific antivirus agents.

The ACE2 peptides described above or elsewhere herein are particularly effective antiviral agents for mono-therapeutic or combined-therapeutic use in treating SARS-CoV infection. One of the benefits of such combination therapies is that lower doses of the other antiviral agents can be administered while still achieving a similar level of antiviral efficacy. Such lower dosages can be particularly advantageous for drugs known to have genotoxicity and mitochondria! toxicity (for example, some nucleoside analogues). Conversely, greater efficacy might be achieved using therapeutic doses of two drugs than could be achieved using only a single drug.

### Anti-Viral Agents

The antiviral agent is suitably selected from antimicrobials, which include without limitation compounds that kill or inhibit the growth of microorganisms (including viruses), and antiviral drugs.

Illustrative antiviral drugs include abacavir sulphate, acyclovir sodium, amantadine hydrochloride, amprenavir, chloroquine, cidofovir, delavirdine mesylate, didanosine, efavirenz, favipiravir, famciclovir, fomivirsen sodium, foscarnet sodium, ganciclovir, hydroxychloroquine, hydroquinone. indinavir sulphate, lamivudine, lamivudine/zidovudine, lopinavir, nelfinavir mesylate, nevirapine, oseltamivir phosphate, ribavirin, remdesivir, rimantadine hydrochloride, ritonavir, saquinavir, saquinavir mesylate, stavudine, valacyclovir hydrochloride, zalcitabine, zanamivir, and zidovudine.

In some alternative embodiments, the ACE2 peptide may be co-administered with an antimicrobial agent including chloroquine, hydroxychloroquine and/or hydroquinone.

In some of the same embodiments and some alternative embodiments, the antiviral agent comprises a recombinant IFN-γ polypeptide (UniProt Accession No. P01574). In some embodiments of this type, the antiviral agent comprises at least a portion of an IFN-γ polypeptide, or a variant of an IFN-γ polypeptide.

As noted above, the present invention encompasses co-administration of an ACE2 peptide in concert with an additional agent. It will be understood that, in embodiments comprising administration of the ACE2 peptide with other agents, the dosages of the actives in the combination may on their own comprise an effective amount and the additional agent(s) may further augment the therapeutic or prophylactic benefit to the patient. Alternatively, the ACE2 peptide and the additional agent(s) may together comprise an effective amount for preventing or treating the SARS-CoV-2 infection. It will also be understood that effective amounts may be defined in the context of particular treatment regimens, including, e.g., timing and number of administrations, modes of administrations, formulations, etc. In some embodiments, the ACE2 peptide and optionally the antiviral agent are administered on a routine schedule. Alternatively, the antiviral agent may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the ACE2 peptide on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve concurrent administration of the ACE2 peptide and the antiviral agent on a daily basis for the first week, followed by a monthly basis for several months, and then every three months after that. Any particular combination would be covered by the routine schedule as long as it is determined ahead of time that the appropriate schedule involves administration on a certain day.

Additionally, the present invention provides pharmaceutical compositions for reducing or abrogating the uptake of viruses (e.g., a SARS-CoV-2) to a cell of the host, the pharmaceutical compositions comprising an ACE2 peptide and optionally an antiviral agent useful for treating the infection. The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients. Depending on the specific conditions being treated, the formulations may be administered systemically or locally. Techniques for formulation and administration may be found in “Remington's Pharmaceutical Sciences”, Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the active agents or drugs of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Dosage forms of the drugs of the invention may also include injecting or implanting controlled releasing devices designed specifically for this purpose or other forms of implants modified to act additionally in this fashion. Controlled release of an agent of the invention may be achieved by coating the same, for example, with hydrophobic polymers including acrylic resins, waxes, higher aliphatic alcohols, polylactic and polyglycolic acids and certain cellulose derivatives such as hydroxypropyl methyl cellulose. In addition, controlled release may be achieved by using other polymer matrices, liposomes or microspheres.

The drugs of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of an active agent, which achieves a half-maximal inhibition in activity of an ACE2 peptide). Such information can be used to more accurately determine useful doses in humans.

Toxicity and therapeutic efficacy of such drugs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50:ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition (see, for example, Fingl et al., 1975, in “The Pharmacological Basis of Therapeutics”, Ch. 1).

Alternately, one may administer the compound in a local rather than systemic manner, for example, via injection of the compound directly into a tissue, which is preferably subcutaneous or omental tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

### Determination of PTM Sites on ACE2

A series of protein domains within both the ACE2 protein were identified as being critical for the entry of the SARS-CoV-2 into the cell. These protein domains are subject to epigenetic post-translational modification (lysine methylation, de-methylation, sumoylation and phosphorylation).

Accordingly, bioinformatic analysis was used to identify specific post-translational modifications (PTMs) that are unique for LSD1 or PKCtheta, and E3 ligase. Multiple studies have now demonstrated that post-translational PTM is a critical and common mechanism for regulating the dynamic regulation of key proteins, including p53.

It was therefore hypothesised that these ACE2 PTMs are critical for interaction with SARS-CoV-2 and viral entry to the cell. Part of the replicative process of viruses include trafficking proteins into the nucleus to employ them as transcriptional regulators for more efficient transcription. Accordingly, putative nuclear localisation signal (NLS) within the ACE2 protein was identified. This peptide was selective and specific for the targeted proteins as well as being selective for the specific domains within each protein.

Extensive bioinformatic sequence analysis was employed using software designed to analyse and identify post-translation motifs within the protein sequence (phosphorylation, acetylation, methylation, glycosylation, ubiquitination, and so on), bioinformatic software to identify nuclear localisation sequences (NLS) were also employed which scored the probability of canonical and non-canonical NLS within the protein sequence. All analysis included cut-off values to reduce false positives and increase stringency. The software employed included NLS-mapper to identify NLS motifs (Kosugi, et al., 2008; Kosugi, et al., 2009a; Kosugi et a., 2009b), PSSMe was used to identify potential sites of methylation/demethylation (Wen et al., 2016), Phosphorylation NetPhos 3.1 Server was used to identify potential phosphorylation motifs (Blom et al., 2004) and Predict-Protein was used for further protein domain analysis (Su et al., 2019; Ofran et al., 2007).

This information was overlayed with the known protein domains within each analysed protein. Finally, this information was checked by protein chemists to finalize peptide inhibitor sequences and targets.

We have identified with the ACE2 protein sequence a series of key serin residues that are phosphorylated by PKCq and key lysine methylation sites, these methylated lysine residues also represent sites of LSD1-mediated de-methylation. Other proteins have been demonstrated to be dynamically regulated by lysine methylation and demethylation or phosphorylation include p53. LSD1 regulates p53 at a single lysine residue conferring exquisite regulatory control on p53 (Huang et. al., 2007). Therefore, all such PTM sites have the potential to significantly influence the regulation of these proteins.

### Example 2

### LSD1 and ACE2 Associate on Cell Surface

LSD1 is a key eraser enzyme, that demethylates key histone proteins and key proteins such as transcription factors whereby this demethylation/methylation post-translational modification has resulted in induction, inhibition or stabilization of the expression of the targeted proteins, such as p53. Based on these data, the role of LSD1 as a key regulator of the receptor ACE2 and TMPRSS2 responsible for shuttling SARS-CoV-2 into the cell was investigated.

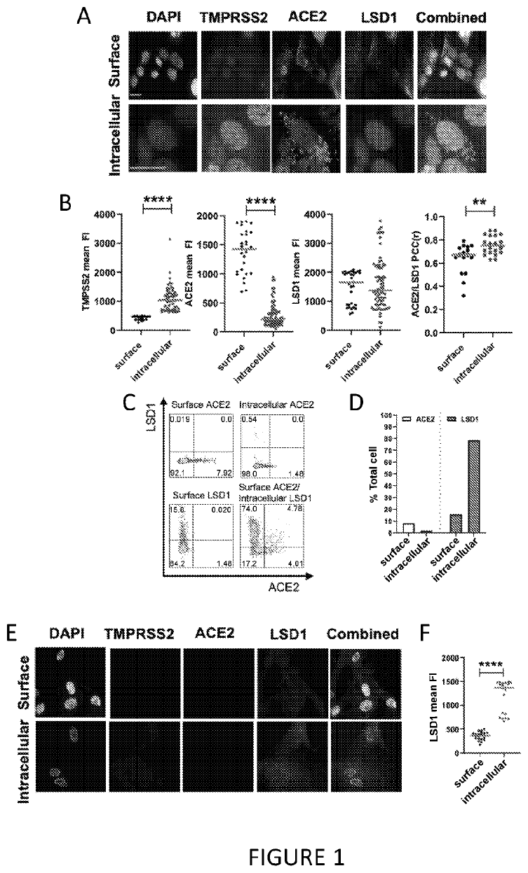

ASI digital pathology analysis was used to examine non-permeabilized Caco-2 cells which monitor cell surface expression and permeabilized cells that monitor intracellular compartmentalisation. Cells were stained positive for the proteins ACE2, TMPRSS2 and LSD1. Strikingly, LSD1, which is traditionally described as a cytoplasmic or nuclear protein, also stained positive on the cell surface (see, FIG. 1). LSD1 significantly co-localized with ACE2 as demonstrated by the PCC(r) co-efficient which adjudicates the degree of co-localisation between two protein targets. This analysis shows strong co-localisation between ACE2 and LSD1 on the cell surface. This was further validated by FACS analysis of Caco-2 cells. MRC5 cells, resistant to SARS-CoV-2 infection do not express ACE2 or TRMPSS2. However, these cells express LSD1 on the cell surface, albeit at 4-fold less compared to SARS-CoV-2 susceptible cell line Caco-2.

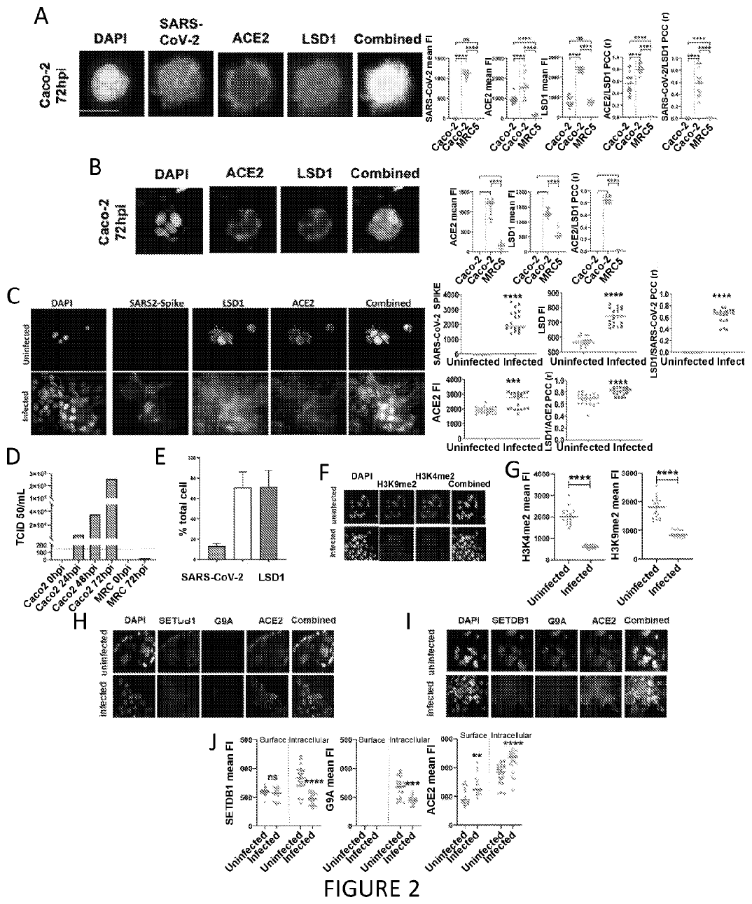

The present inventors then investigated the effect of SARS-CoV-2 infection on the LSD1 and ACE2/TRMPSS2 co-expression. High resolution quantitative imaging and FACS analysis was used to examine Caco-2, or Caco-²/ₐMRC5 cells infected with SARS-CoV-2. Cells were stained with ACE2; and the epigenetic enzyme LSD1; or LSD1 and antibodies for the nucleocapsid or spike protein of SARS-CoV-2 (see, FIG. 2A-C). LSD1 significantly co-localized higher with ACE2 in cells infected with SARS-CoV-2, as compared to ACE2/LSD1 expression in uninfected CaCo2 and MRC5 cells (which are not susceptible to SARS-CoV-2 infection) as demonstrated by the PCC(r) co-efficient which adjudicates the degree of co-localisation between two protein targets, as well as significantly upregulated expression of both LSD1 and ACE2 was demonstrated in infected cells. MRC5 had no expression of ACE2 and no staining of virus proteins (FIG. 2D). Interestingly, LSD1 co-localized with both SARS-CoV-2 spike protein and the nucleocapsid proteins. Furthermore, LSD1 nuclear activity was increased as measured by reduction in expression of H3k9me2 and H3k4me2 (FIG. 2F, G). Strikingly, when the two key methyl transferases (G9A and SETDB1) were compared, there was either little expression on the cell surface or no increase following SARS-CoV-2 infection (FIG. 2H-J).

These results clearly demonstrate that LSD1 and ACE2 have increased association on the cell surface.

### Materials and Methods

### Microscopv Methods

To examine the signature of LSD1, ACE2, TMPRSS2 and SARS-CoV-2 in infected cells or uninfected cells (untreated or treated with MAOis or EPI-111 (myristyl-RRTSRRKRAKV-OH) [SEQ ID NO: 37] Caco-2 or MRC5 cells were permeabilised by incubating with 0.5% Triton X-100 for 15 min, blocked with 1% BSA in PBS and were probed with either LSD1 (Rabbit host), ACE2 (conjugated to AF594), TMPRSS2 (Mouse host) and in the case of infected cells SARS-CoV-2 (Mouse Host) and visualized with a donkey anti-mouse AF 488 or donkey anti-rabbit 647 or the antibodies were primary conjugated to an appropriate AF fluorochrome (AF 594). Cover slips were mounted on glass microscope slides with ProLong Glass Antifade reagent (Life Technologies). Protein targets were localised by digital pathology laser scanning microscopy. Single 0.5 pm sections were obtained using a ASI Digital pathology microscope using 100× oil immersion lens running ASI software. The final image was obtained by averaging four sequential images of the same section. Digital images were analyzed using automated ASI software (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Ddigital images were also analysed using ImageJ software (ImageJ, NIH, Bethesda, MD, USA) to determine the total cell fluorescence or cell surface only fluorescence for non-permeabilised cells. Digital images were analysed using ImageJ software (ImageJ, NIH, Bethesda, MD, USA) to determine the either the Total Nuclear Fluorescent Intensity (TNFI), the Total Cytoplasmic Fluorescent Intensity (TCFI). ImageJ software with automatic thresholding and manual selection of regions of interest (ROls) specific for cell nuclei was used to calculate the Pearson's co-efficient correlation (PCC) for each pair of antibodies. PCC values range from: −1=inverse of co-localisation, 0=no co-localisation, +1=perfect co-localisation. The Mann-Whitney nonparametric test (Graph Pad Prism, Graph Pad Software, San Diego, CA) was used to determine significant differences between datasets.

### Example 3

### LSD1 Inhibitors Abrogate ACE2 Expression and Inhibit SARS-CoV-2 Expression

Based on the above findings, it was hypothesized that LSD1 complexes with and de-methylates ACE2 to stabilize expression.

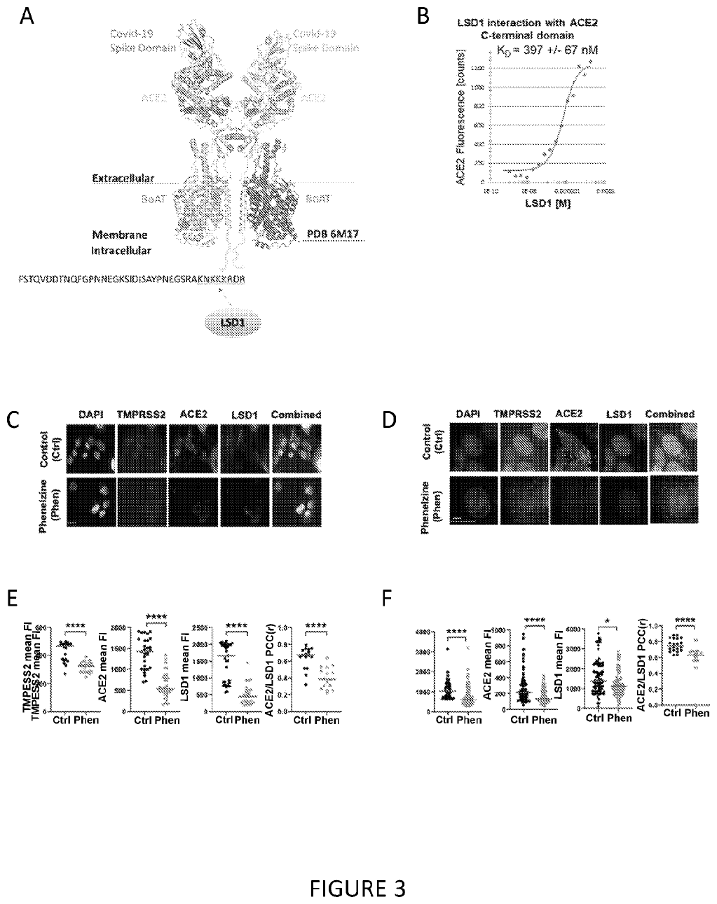

Bioinformatic analysis clearly shows that in ACE2 there are three high probability lysine residues for post-translation modification by LSD1 and that these lysine residues are part of the C-terminal domain and a novel, putative nuclear localization sequence (NLS). This C-terminal domain of ACE2 is highly flexible, disordered domain suitable for protein-protein interactions (see, FIG. 3A). The present inventors established in vitro with a recombinant LSD1 and ACE2 C-terminal domain that indeed they directly interacted at a 1:1 ratio (see, FIG. 3B). Next, the present inventors demonstrated that inhibition of LSD1 with phenelzine (a dual targeting demethylase and nuclear LSD1 inhibitor) abrogated expression and co-localization of LSD1 with ACE2 and TMRPSS2 (FIG. 3C-F). Furthermore, there was minimal effect on ACE2 transcription. These results show that LSD1 inhibition interferes via inhibiting the demethylation de-stabilizes ACE2 protein expression on the cell surface in contrast to TMPRSS2 which is regulated via the traditional role of LSD1 as an epigenetic regulator.

### Materials & Methods

### Microscale Thermophoresis Methods

Binding affinity measurements were performed on a Monolith NT.115 (NanoTemper Technologies). The fluorescein-Ahx tagged ACE2 peptide sequence RDRKKKNKARSGEN was manufactured by Genescript [SEQ ID NO: 38]. Each reaction consisted of 10 μL of the labelled peptide at 444 nM, mixed with unlabelled LSD1 at the indicated concentrations. All experiments were measured at 25° C. with laser off/on/off times of 5/30/5 s. Experiments were conducted at 20% light-emitting diode power and 20-40% MST infra-red laser power. Data from three independently performed experiments were fitted to the single binding model via the NT. Analysis software version 1.5.41 (NanoTemper Technologies) using the signal from Thermophoresis+T-Jump.

### Example 4

### Global Transcript Analysis

In order to identify unbiased global gene expression programs impacted by LSD1 inhibition in SARS-Cov-2 infected CaCo2 cells, global RNA sequencing analysis was undertaken to allow the identification of such gene clusters.

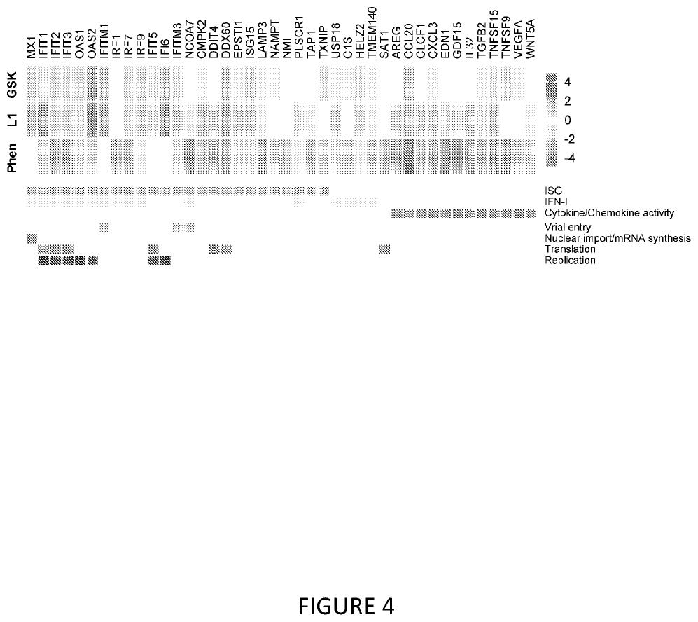

LSD1 inhibition albeit at different degrees, impacts on key anti-viral processes, key proteins responsible for viral entry and the transcription and replication of the SARS-CoV-2 virus in the host cell (see, FIG. 4). The different degrees of impact on these pathways by the LSD1 inhibitors can be attributed to the different modes of action of each inhibitor, with phenelzine impacting on both catalytic, nuclear and structural functions.

### Example 5

### Interplay of Intracellular ACE2 in Infected Cells

The signature of intracellular ACE2 was examined in infected cells, including nuclear and cytoplasmic fractions of ACE2, to understand the role of ACE2 in SARS-CoV-2 infection.

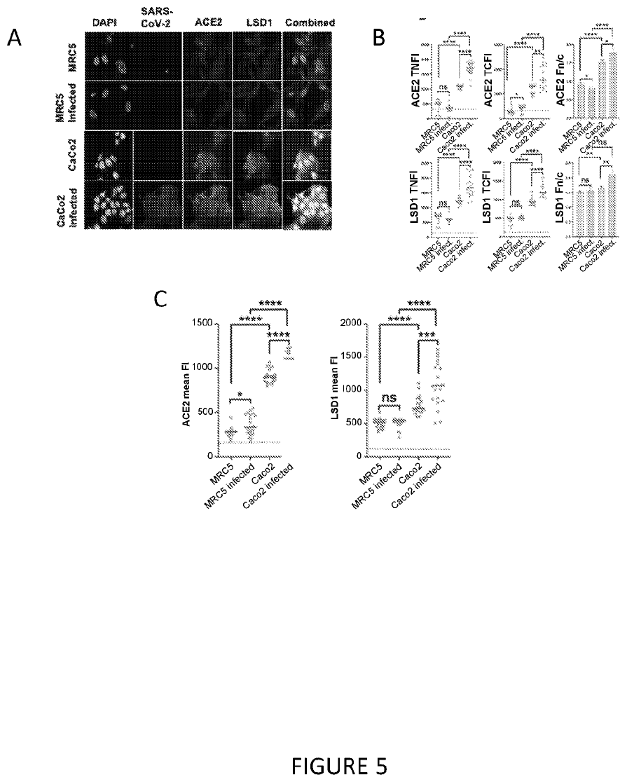

Caco-2 cells susceptible to SARS-CoV-2 infection displayed increased cytoplasmic and nuclear expression of ACE2 in permeabilized cells (FIGS. 5A-C). The Fn/c ratio (a score >than 1 indicates nuclear bias) of ACE2 also increased significantly upon infection. A similar pattern is observed for LSD1.

### Materials & Methods

### RNA Sea Analysis

RNA-seq data were obtained from Caco-2 cell line infected with SARS-CoV-2. Three different treatments were tested (named Phe, Gsk, and L1), with each of the treatments targeting the same gene but in different ways. A total of 8 samples from four experimental groups were collected:
Caco-2 cells infected with SARS-CoV-2 control (2× replicates);
Caco-2 cells infected with SARS-CoV-2 treated with Phe (2× replicates);
Caco-2 cells infected with SARS-CoV-2 treated with Gsk (2× replicates); and
Caco-2 cells infected with SARS-CoV-2 treated with L1 (2× replicates).

The aim was to perform differential expression analysis using edgeR between the control group and each of the treated groups to find the differentially expressed genes. The present inventors then compared the genes between the treatment groups to find common and unique genes, and also perform pathway analysis.

RNA-seq data were generated, fastq data were downloaded to the QIMR Berghofer Medical Research Institute server, and then archived to the HSM by Scott Wood. Sequence reads were trimmed for adapter sequences using Cutadapt (version 1.9; Martin (2011)) and aligned using STAR (version 2.5.2a; Dobin et al. (2013)) to the GRCh37 assembly with the gene, transcript, and exon features of Ensembl (release 89) gene model, and the SARS-CoV-2 RefSeq accession NC 045512. Quality control metrics were computed using RNA-SeQC (version 1.1.8; DeLuca et al. (2012)) and expression was estimated using RSEM (version 1.2.30; Li and Dewey (2011)).

### Quality Control

The quality control of RNA-seq samples is an important step to guarantee quality and reproducible analytical results. RNA-SeQC was run for this purpose, the results of which can be found on the HPC cluster. Another common quality metric is whether the RNA sample is contaminated with mitochondrial DNA (mtDNA) or whether there is a high amount of ribosomal RNA (rRNA) in the sample. We determined the number of reads which mapped to Ensembl biotypes, including protein-coding genes, rRNA, and mitochondria. Given we use a threshold of 95% of reads mapping to protein coding regions, 7 samples passed this QC measure.

### Normalization

The aim of normalisation is to remove differences between samples based on systematic technical effects to warrant that these technical biases have a minimal effect on the results. The library size is important to correct for as differences in the initial RNA quantity sequenced will have an impact on the number of reads sequenced. Differences in RNA sequence composition occurs when RNAs are over-represented in one sample compared to others. In these samples, other RNAs will be under-sampled which will lead to higher false-positive rates when predicting differentially expressed genes.

### Normalisation Methods

In our analysis, we corrected for library size by dividing each sample's gene count by million reads mapped. This procedure is a common approach known as counts per million (CPM). We further corrected for differences in RNA composition using a method proposed by Robinson and Oshlack (2010a) called trimmed mean of M values (TMM). We used the function calcNormFactors( ) from the edgeR package (Robinson, McCarthy, and Smyth (2010b)) to obtain TMM factors and used these to correct for differences in RNA composition.

### Differential Expression Analysis

Differential expression (DE) analysis was performed using the R package edgeR (Robinson, McCarthy, and Smyth (2010b)). Note that the inputs for DE analysis are the filtered but not normalised read counts, since edgeR performs normalisation (library size and RNA composition) internally. The gImQLFit( ) function was used to fit a quasi-likelihood negative binomial generalised log-linear model to the read counts for each gene. Using the gImQLFTest( ) function, we conducted gene-wise empirical Bayes quasi-likelihood F-tests for a given contrast. As per the edgeR user's guide, “the quasi-likelihood method is highly recommended for differential expression analyses of bulk RNA-seq data [versus the likelihood ratio test] as it gives stricter error rate control by accounting for the uncertainty in dispersion estimation.”

### Example 6

### Novel P604 Ace2 Peptide Inhibitor

After determining that the C-terminal domain of ACE2 appears to be critical site mediated by LSD1 demethylase activity for ACE2 stability on the cell surface, the present inventors proposed developing a competitive peptide inhibitor that interferes and blocks LSD1 targeting this site, which would abrogate ACE2 expression. Furthermore, the present inventors proposed that the nuclear localisation sequence (NLS) (i.e., RKKKNK; SEQ ID NO: 48) in the C-terminal domain is a site for binding by IMPa, a key nuclear shuttling protein. It was hypothesised that interaction of the IMPα polypeptide at this site is enhanced by demethylation will allow translocation of ACE2 and any bound virus to the nuclear of the cell. The present inventors also considered that ACE2 has a novel nuclear role in directly regulating transcription akin to that now identified for key signal kinases traditionally functioning as cytoplasmic proteins.

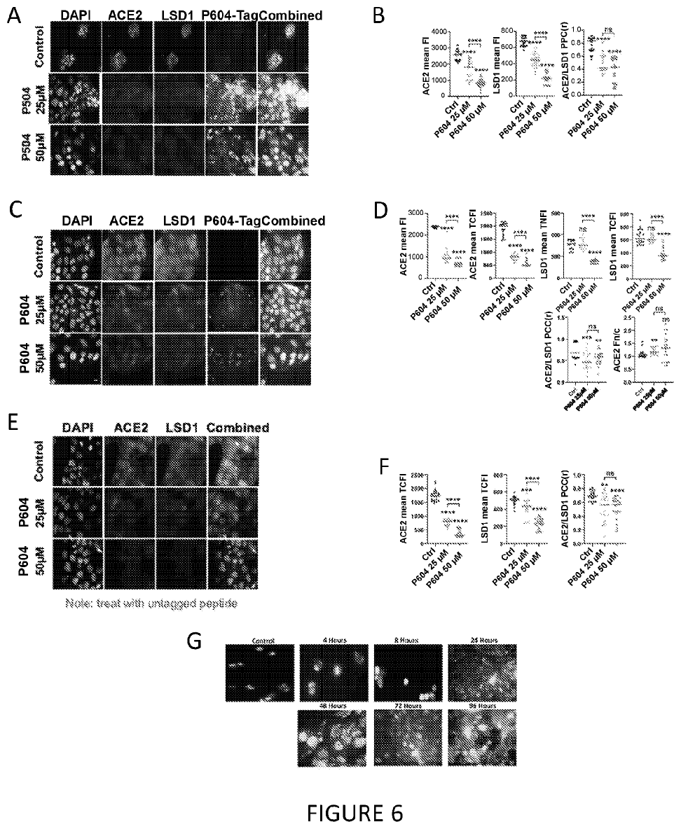

A peptide sequence (P604) was constructed to target the LSD1-mediated demethylation motif and NLS on the C-terminal domain of ACE2 (TGIRDRKKKNKRS; SEQ ID NO: 3). It is also shown this domain interacts with IMPa. Caco-2 cells treated with ACE2 peptide targeting the interaction domain of LSD1 and ACE2 (which is also the putative NLS of ACE2) de-stabilizes ACE2 expression on the cell surface (see, FIG. 6), inhibiting the expression of ACE2 and as a result reducing cell surface LSD1 expression (see, FIGS. 6A-F). The PCC(r) of LSD1 and ACE2 was also significantly abrogated, which measures the degree of co-localisation between two protein markers (see FIG. 6).

### Example 7

### Effect of P604 ACE2 Peptide Inhibitor on SARS-COV-2

The present inventors then investigated whether the effect of the P604 ACE2 peptide inhibitor impacted on expression of host ACE2 gene or TMPRSS2 gene and the Spike protein of SARS-CoV-2, or the expression of the nucleocapsid of SARS-CoV-2.

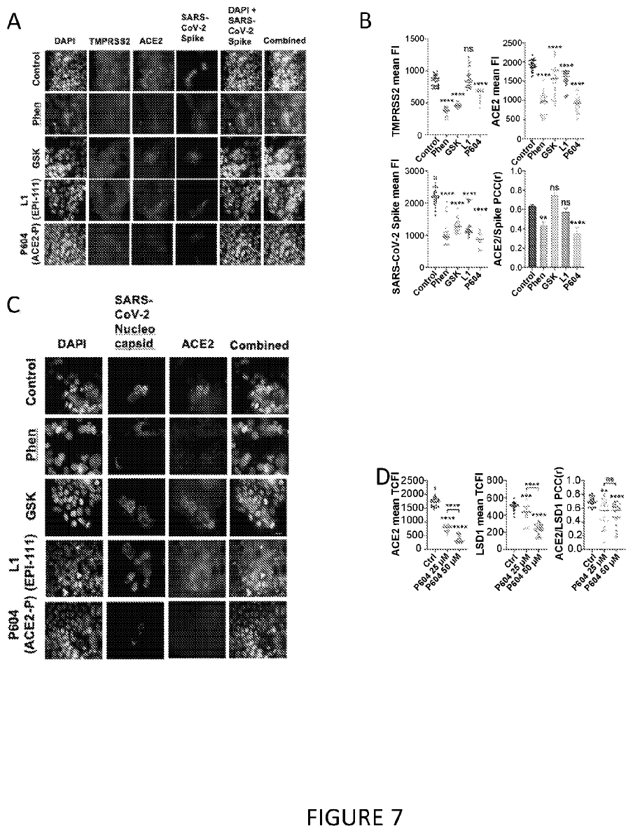

FIG. 7 shows that the ACE2 peptide inhibitor (P604) was able to significantly inhibit and downregulate the expression of both host ACE2, and the nucleocapsid and Spike proteins of SARS-CoV-2.

### Example 8

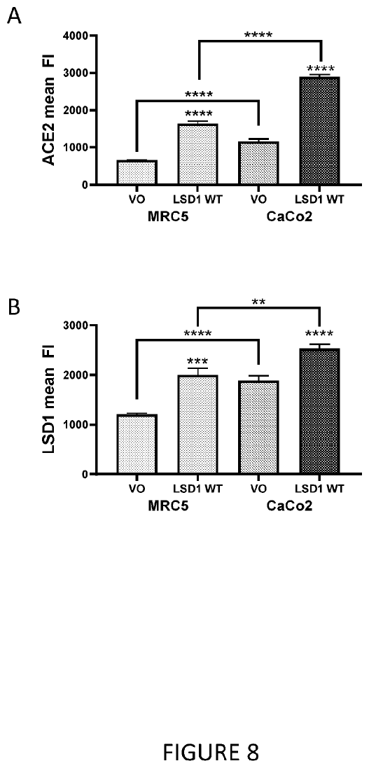

MRC5 or Caco-2 cells were transfected with either VO (Plasmid Vector Only) or LSD1-WT (Plasmid Vector with LSD1 WT gene) using the Neon transfection system. Immunofluorescent analysis was carried out with antibodies against either ACE2 or LSD1. As expected in light of the data presented above, there was also a significant increase of LSD1 in cells transfected with LSD1-WT compared to VO (FIG. 8). Analysis revealed that overexpression of LSD1 in cells transfected with LSD1-WT significantly increased expression of ACE2 in Caco-2 cells as well as strikingly increased expression of ACE2 in MRC5 cells.

### Materials & Methods

Caco-2 or MRC5 cells were transfected with LSD1 WT plasmid or VO constructs using the NEON electroporation transfection system (Life Technologies). Transfected cells were permeabilised by incubating with 0.5% Triton X-100 for 15 min and were probed with a rabbit anti-LSD1 and mouse anti-ACE2 antibodies. Cover slips were mounted on glass microscope slides with ProLong NucBlue Antifade reagent (Life Technologies). Protein targets were localised by confocal laser scanning microscopy. Single 0.5 pm sections were obtained using an ASI Digital pathology platform using 100× oil immersion lens running ASI software. The final image was obtained by averaging four sequential images of the same section. Digital images were analysed using ImageJ software (ImageJ, NIH, Bethesda, MD, USA) to determine the mean Fluorescent Intensity (mean FI). The Mann-Whitney nonparametric test (GraphPad Prism, GraphPad Software, San Diego, CA) was used to determine significant differences between datasets.

### Example 9

Collection and Storage of BALCS, PBMCs and Plasma from SARS-Cov-2 Patients

Bronchoalveolar Lavage cells (BALC) and Peripheral Blood Mononuclear Cells (PBMC) collection and storage; plasma collection, SARS-CoV2 virus detection, and secure storage.

### Materials & Methods

Patients with SARS-CoV-2 infection only (cohort 1, n=5); early and advanced solid tumour cancer patients with SARS-CoV-2 infection (cohort 2, n=5); and healthy donors (cohort 3, n=5). Written consent will be obtained for study participation and patients will be followed up as per standard national/local guidelines with regular clinical examination. Blood samples (40 mL total) will be collected by a clinical trial nurse as part of standard blood collection. Clinicopathological/virological data will be collected by the clinical team. PBMCs will be isolated according to our established protocols and stored in liquid nitrogen. Plasma will be collected for SARS-CoV-2 detection by RT-PCR and stored at 80° C. for the virus infection assay. BALF (20 mL/patient) will be obtained and processed within 2 hours in a BSL-3 laboratory. BALCs will be isolated by filtering and centrifugation before being resuspended in medium for future use.

SARS-CoV-2-infected cells with/without inhibitor treatments will be assayed by qRT-PCR for ACE2 and flow cytometry and digital pathology using antibodies targeting ACE2.

PBMCs will be pre-treated with inhibitors and killing assays performed using the xCELLigence® Real Time Cell Analyzer.

### Example 10

### Novel Cell Permeable Peptides Inhibit ACE2-Spike Interactions and Cellular Entry of SARS-COV-2

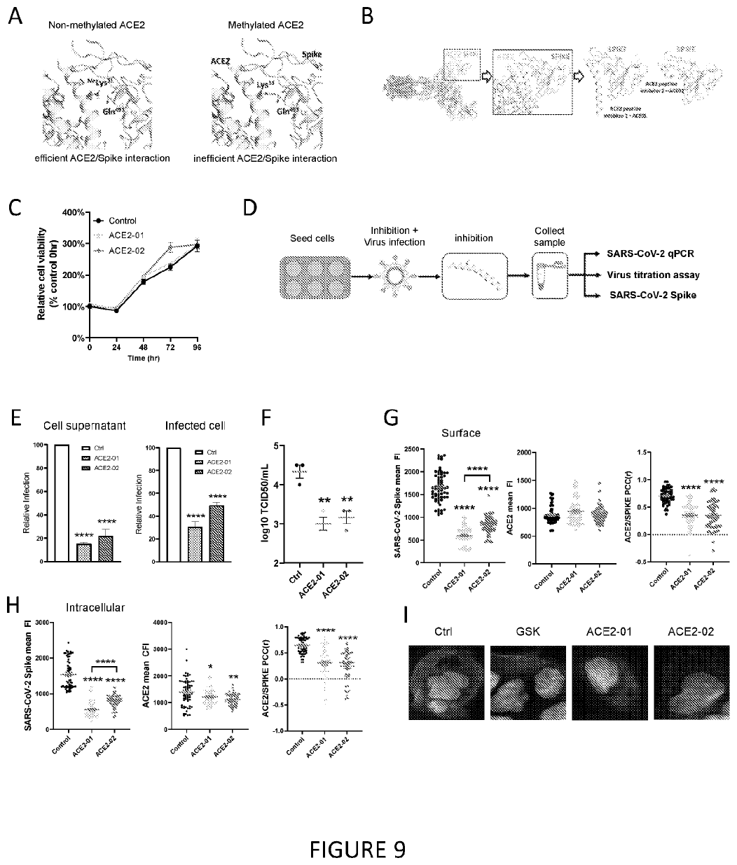

To examine the effect of interactions between demethylated ACE2 lysine 31 and SARS-CoV-2 spike protein at the cell surface, two novel ACE2 peptide inhibitors were developed (ACE2-01 [SEQ ID NO: 24] and ACE2-02 [SEQ ID NO: 26], see Table 5) through structural analysis and modelling (FIG. 9A) of the target sequence, peptide length optimization, and alanine walking to identify critical residues. Overlapping the spike interaction region, ACE2-01 extends downstream past the lysine demethylation motif, while ACE2-02 extends and terminates at lysine 31 to facilitate studies of this critical residue (FIG. 9B). Both peptides are predicted to competitively block interactions between the spike protein and lysine 31, either by interfering with ACE2/spike interactions or by binding to the spike protein as a decoy. These peptides also competitively block enzymatic access to lysine 31 as a decoy interaction, interfering with lysine 31 demethylation by mimicking the ACE2/spike binding domain/lysine d-methylation motif, meaning that the LSD1 catalytic pocket or the RBD spike domain interact with the peptide and not the target protein to prevent lysine 31 demethylation or spike-ACE2 interactions.

TABLE 5
NOVEL ACE2 PEPETIDE SEQUENCES

| | |
|---|---|
| ACE2-01 | IEEQAKTFLDKFNHEAEDLFYQSSLASWNYNT |
| | [SEQ ID NO: 49] |
| ACE2-02 | STIEEQAKTFLDK [SEQ ID NO: 50] |

In comparison to untreated control cells, neither ACE2 peptide inhibitor altered cell proliferation up to 96 hours of treatment (FIG. 9C). To assess the impact of ACE2-01 and ACE2-02 on SARS-CoV-2 replication, Caco-2 cells were infected with SARS-CoV-2 (MOI 1.0) and then treated with the peptide inhibitors for 48 hours (FIG. 9D). Using qRT-PCR of both the culture supernatants and infected cells, ACE2-01 and ACE2-02 treatment significantly reduced infection by 6.6-fold and 4.6-fold, respectively, in cell culture supernatants at 48 hpi (FIG. 9E). Viral RNA also decreased by 3.3-fold and 2-fold in infected cells following ACE2-01 and ACE2-02 treatment at 48 hpi, respectively (FIG. 9E).

Next, infectious viral titers were quantified by median tissue culture infectious dose (TC1 D₅₀) of supernatants from infected cells treated with ACE2-01 and ACE2-02, which further confirmed reductions in viral load by 4.5-fold and 3.2-fold, respectively (FIG. 9F). Furthermore, inhibition of SARS-CoV-2 infection was assessed using digital pathology to detect spike protein intensity (FIGS. 2G and 2H). Both inhibitors significantly reduced SARS-CoV-2 spike protein at the cell surface and intracellularly in infected cells (FIGS. 2G and 2H), with co-localization of spike and ACE2 also significantly reduced (FIG. 2G). Finally, a proximity ligation assay was used to assess the co-localization of ACE2 and spike protein at the surface of SARS-CoV-2 infected Caco-2 cells. GSK treatment significantly decreased interaction between ACE2 spike protein, and ACE2-01 and ACE2-02 peptide inhibitors further disrupted ACE2/spike complexes at the cell surface (FIG. 21). This suggests that methylation of ACE2 via inhibition of LSD1 activity contributes to blocking access to ACE2 by the SARS-CoV-2 spike protein. Furthermore, competitively blocking access to the lysine 31 motif with peptide inhibitors inhibits spike protein access to ACE2. Taken together, these data demonstrate that interactions between the viral spike protein and the lysine 31 demethylation motif of ACE2 are important for SARS-CoV-2 replication, and our peptide inhibitors have antiviral activity by significantly reducing spike protein co-localization with ACE2.

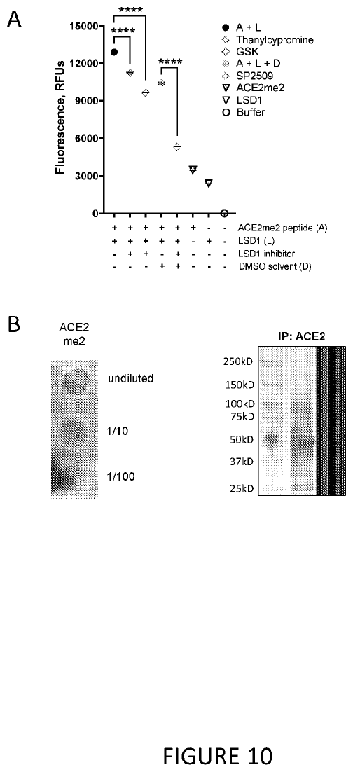

The above data show that LSD1 associates with ACE2 at the cell surface following SARS-CoV-2 infection. Furthermore, ACE2 lysine 31 is predicted to undergo de-methylation/methylation (FIG. 10A) and interact with glutamine 493 in the SARS-CoV-2 spike protein receptor-binding domain (RBD) (Shang et al., 2020). The present inventors therefore addressed the ability of LSD1 to directly de-methylate ACE2 at lysine 31 with LSD1 activity assays using peptides mimicking the methylated lysine 31 motif. To assess whether LSD1 inhibition reduced ACE2 demethylation at lysine 31, recombinant LSD1 protein alone or pre-incubated with di-methylated ACE2 peptide (Table 6 and FIG. 10B) was used as a substrate to measure the demethylation reaction with an in vitro LSD1 activity assay. The peptide contained the motif predicted to undergo methylation/de-methylation using the in silico prediction software PSSme (Sheng et al., 2018) and also representing the binding region between glutamine 493 in the receptor-binding domain (RBD) of the SARS-CoV-2 spike protein and ACE2 (Shang et al., 2020): QAKTFLD{Lys(Me2)}FNHEAED [SEQ ID NO: 39], with a di-methylated lysine at position 31. LSD1 efficiently de-methylated the ACE2 peptide at lysine 31.

TABLE 6
SUMMARY OF ACE2ME2 PEPETIDE SEQUENCES

| ACE2 | | |
|---|---|---|
| Peptide | Sequence | Length |
| ACE2me2 | QAKTFLD{Lys(Me2)}FNHEAED [SEQ ID | 15 |
| | NO: 39] | |
| PEP1 | TGIRDR{Lys(Me2)}KKNKARS [SEQ ID | 14 |
| | NO: 40] | |
| PEP2 | TGIRDRK{Lys(Me2)}KNKARS [SEQ ID | 14 |
| | NO: 41] | |
| PEP3 | TGIRDRKK{Lys(Me2)}NKARS [SEQ ID | 14 |
| | NO: 42] | |
| PEP3 | TGIRDR{Lys(Me2)}K{Lys(Me2)}NKARS | 14 |
| | [SEQ ID NO: 43] | |

### Example 11

### Alanine Mutagenesis of ACE2 Peptide Inhibitors

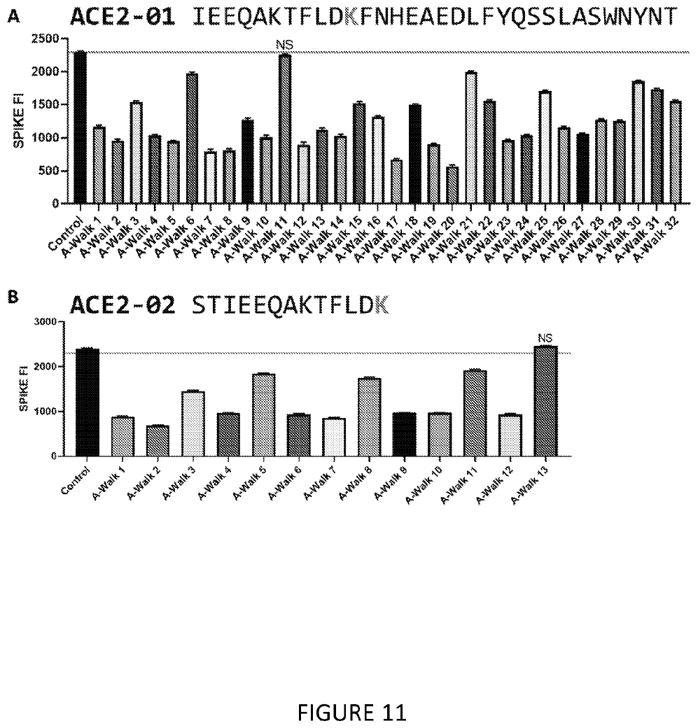

The above data clearly demonstrates that both ACE2-01 and ACE2-02 peptides were able to significantly inhibit Spike protein binding to the cell surface of CaCo2 cells. Accordingly, the inventors were next motivated to investigate the essential residues of the tested peptide inhibitors. Alanine mutagenesis walk experiments revealed that substitution of an Alanine at positions Lysine 31 significantly reduced the effectiveness of inhibition indicating that this lysine residue is critical (FIG. 11). While overall no other alanine substitutions had a similar effect, alanine substitution at Lysine 26 reduced the effectiveness of the peptides as compared to other substitutions (but did not eliminate the inhibition).

From the overlapping sequence between the two peptide inhibitors and based on virus infection work, this demonstrates that the shorter peptide (ACE2-02) is as effective as the longer peptide sequence (ACE2-01). As long as overall charge/size is preserved for the other residues there is no obvious effect on inhibition efficacy.

### Materials & Methods

Caco-2 cells (8×10⁴) were seeded on coverslips for 48 hours before treatment with peptides from an alanine walk for ACE2-01 or ACE2-02 (10 mM for each peptide) for 24 hours followed by treatment with 20 μL of purified SARS-CoV-2755 spike protein 51 (Glu14-Ser680) containing a poly-histidine tag at the C-terminus (1.52 mg/mL) for 24 hours. Non-permeablized samples were then stained with antibodies specific for the SARS-CoV-2 spike protein and visualized with a secondary antibody targeting the host primary antibody. Protein targets were localized by digital pathology laser scanning microscopy. Single 0.5 pm section were obtained using an ASI Digital pathology (ASI Digital pathology is characterization of both the fluorescent intensity as per normal immunofluorescent imaging as well as the ability to count the population of cells positive or negative for antibodies, allow population dynamics to be investigation using powerful custom designed algorithms and automated stage. This also allows the imaging and counting of large cell numbers for statistical power) microscope using a 100× oil immersion lens running ASI software. The final image was obtained by averaging four sequential images of the same section. Digital images were analyzed using automated ASI software as described previously 63 (Applied Spectral Imaging, Carlsbad, CA) to determine the distribution and intensities automatically with automatic thresholding and background correction of the average fluorescence intensity (FI), allowing for the specific targeting of expression of proteins of interest.

### Example 12

### P604 ACE2 Pepetide Inhibitor Disrupts Nuclear ACE2 Importin Machinery

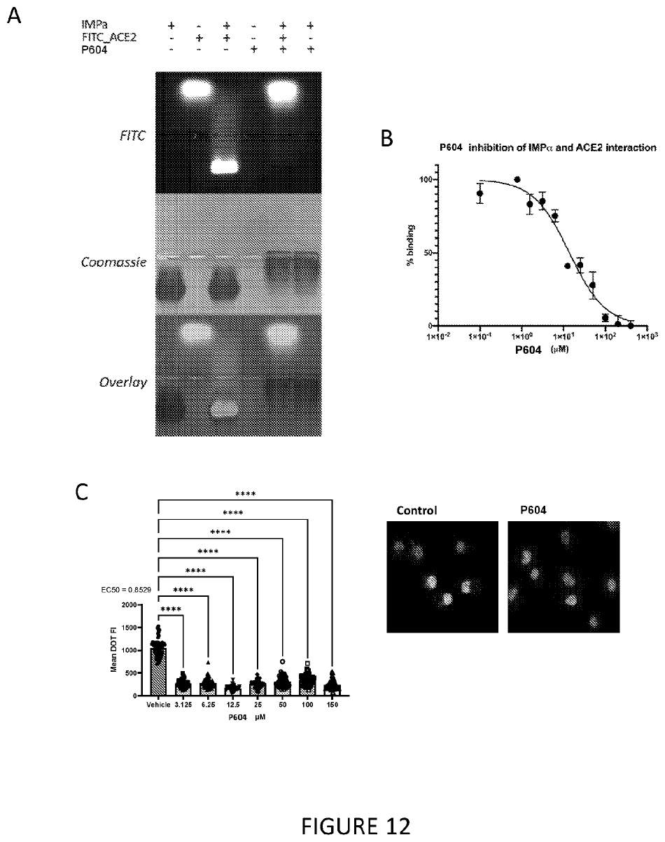

The P604 ACE2 peptide inhibitor inhibits the nuclear shuttling of the ACE2 via directly inhibiting the ACE2-importin complex in vitro (FIG. 12A, B) and in SARS-CoV-2 susceptible cell lines (FIG. 12C, D). Importantly, these data confirm that this peptide inhibitor specifically targets nuclear ACE2 and does not impact on other critical nuclear proteins targeted by the importin pathway. DUOLINK analysis which detects the close interaction of two protein targets, unmodified ACE2 (ACE2unmod) or IMP1, was performed on H1299 (lung cell line) treated with either control or increase concentrations of the P604 peptide. Analysis revealed that in the vehicle control samples ACE2 and IMP1 formed a significant interaction indicating that ACE2unmod is able to strongly interact with the importin nuclear transport machinery. Strikingly even the lowest concentration of the P604 peptide significantly all but abrogated this interaction (FIG. 12C). Collectively with the examples described above, the present inventors clearly show that the P604 ACE2 peptide is a selective inhibitor of nuclear ACE2-importin pathway, a critical pathway for SARS-Cov2 replication.

### Materials & Methods

### Fluorescence Polarization Competition Assays.

Fluorescence polarization assays were performed using the CLARIOstar Plus plate reader (BMG Labtech) with the fluorescein-Ahx-tagged ACE2 peptide sequence RDRKKKNKARSGEN (i.e., residues 776-779 of sequence set forth in SEQ ID NO: 1) manufactured by GeneScript Biotech (Piscataway, NJ) [SEQ ID NO: 38], the P604 ACE2 peptide sequence Myristyl-TGIRDRKKKNKARS-OH manufactured by Mimitopes Pty Ltd (Melbourne, Australia), and recombinantly expressed importin-α ΔI BB protein [SEQ ID NO: 44]. Each assay contained 50 nM ACE2 FITC, 10 pM importin-α ΔI BB protein, and two-fold serially-diluted P604 ACE2 peptide (starting concentration 400 pM) across 10 wells to a total volume of 200 pL. Fluorescence polarization readings were taken using 96-well black Fluotrac microplates (Greiner Bio-One; Kremsmunster, Austria). Assays were repeated in triplicate and contained a negative control (no inhibitor) and blank (no importin-α ΔI BB protein). Triplicate data was normalised and fitted to a single inhibition curve using GraphPad Prism.

### Electrophoretic Mobility Shift Assay.

FITC-Ahx-tagged ACE2 peptide (90 pM) was mixed with importin-a Al BB protein (100 pM) and P604 ACE2 peptide inhibitor (500 pM) and electrophoresed through a 1% agarose gel in TB Buffer (45 mM boric acid, 45 mM Tris base, pH 8.5) for 90 min at 40 V. ACE2 peptide alone, P604 ACE2 peptide inhibitor alone and importin-a AIBB alone were used as controls. The gel was first imaged under UV light using a Gel Doc XR+ system before being stained using Coomassie brilliant blue.

### Proximity Ligation Assay.

The DUOLINK proximity ligation assay was employed using PLA probe anti-mouse PLUS (DU092001), PLA probe anti-rabbit MINUS (DU092005), and DUOLINK In Situ Detection Reagent Red Kit (DU092008) (Sigma Aldrich). Cells were fixed, permeabilized, and incubated with primary antibodies targeting ACE2umodified (ACE2unmod) and IMPa1. Cells were processed according to the manufacturer's recommendations. Finally, coverslips were mounted onto slides and examined as above.

### Example 13

### P604 ACE2 Peptide Inhibitor Reduces Lung Inflammation

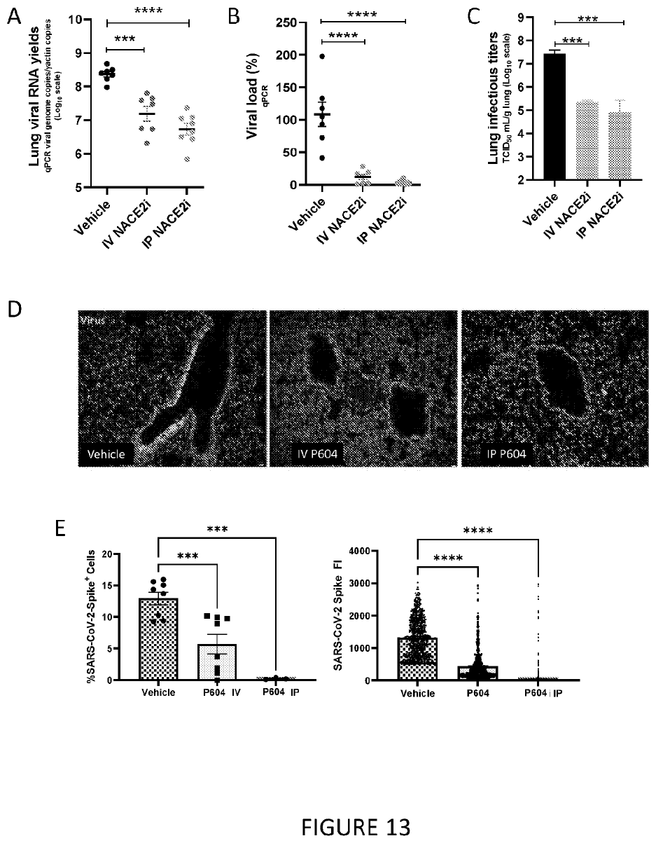

Significant reductions in viral RNA, were observed in the lungs of animals treated with the P604 ACE2 peptide inhibitor (amino acid sequence TGIRDRKKKNKARS [SEQ ID NO: 3]) in a hamster model, administered by IV and IP injection, respectively (FIG. 13A). When normalized to the vehicle group, animals treated with the P604 ACE2 peptide, by both IV and IP administration, showed a substantial decrease in viral load of 88% and 96%, respectively (FIG. 13B). Lung infectious titers, as measured by a TCID₅₀ assay, were reduced for P604 ACE2 peptide IV and IP treated animals, respectively, in comparison to the vehicle group (FIG. 13C). FIG. 13D ASI Digital pathology imaging demonstrated that the population of cells in bronchial lung sections positive for the SARS-CoV-2 spike protein was significantly reduced by P604 ACE2 peptide treatment via either the IV or IP route. Additionally, even where cells were positive for SARS-CoV-2 spike protein, analysis of expression of the Spike protein revealed that overall intensity of signal was significantly reduced as well in both IP and IV administration treatment groups.

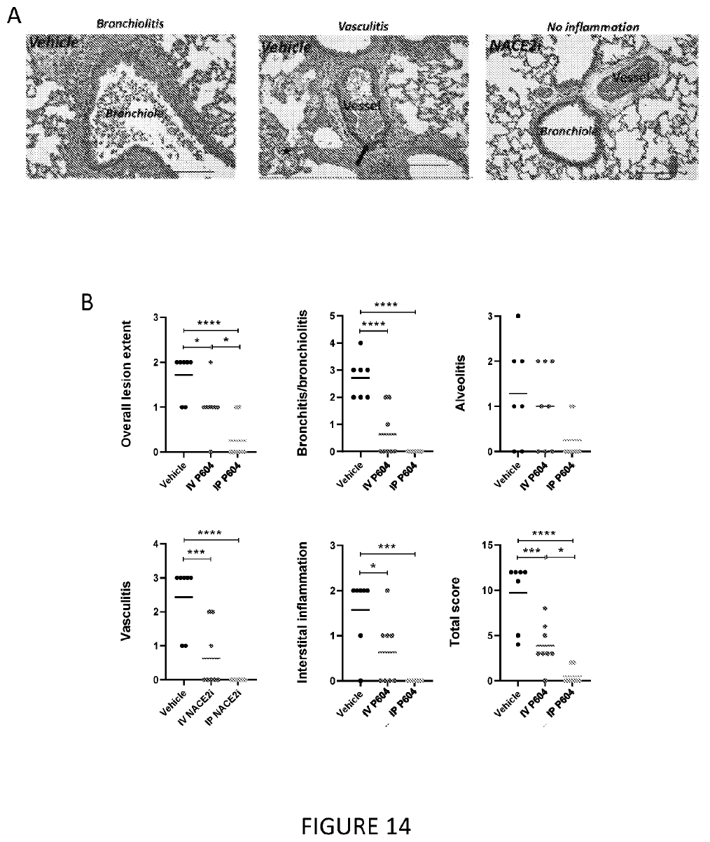

To assess the impact of the P604 ACE2 peptide inhibitor treatment on SARS-Cov-2 induced lung pathology, haematoxylin and eosin (H&E) stained lung sections were scored by a single veterinary pathologist, blinded to the treatments, as previously described (FIG. 14A). Histology scores for the following parameters: overall lesion extent, bronchitis, alveolitis, vasculitis, interstitial inflammation and pneumocyte hyperplasia were scored using a 0-4/0-5 scale and scores for each lung summed to obtain the total histopathological score (FIG. 14B). As lungs were isolated during the early stages of inflammation at 2 dpi, mild-moderate pulmonary changes were observed (FIG. 14A-B), most notably within the bronchioles and no pneumocyte hyperplasia was observed. During this early phase of lung injury, evidence of severe degeneration and necrosis of bronchiolar epithelial cells was visible in the vehicle group with intraepithelial leukocyte infiltration and widespread apoptosis (FIG. 14, first image). Early vascular changes in the vehicle group included margination of heterophils and monocytes followed by transmural migration resulting in disruption of the endothelial lining and the smooth muscle of the media (FIG. 14A middle photo, FIG. 14B). In comparison, the majority of the P604 ACE2 peptide inhibitor IV and IP treated animals displayed minimal changes in the bronchioles or vessels (FIG. 14A, right panel, FIG. 14B). Additionally, early mild alveolar accumulation of heterophils and alveolar macrophages was most consistently seen in the vehicle group. Overall, mean cumulative scores of treated animals were substantially lower than the vehicle group, suggesting that P604 ACE2 peptide inhibitor treatment protects against early lung inflammation associated with SARS-Cov-2 infection.

### Materials & Methods

### Hamster Tolerance and Efficacy Studies.

Female golden Syrian Hamsters (6-8 weeks) were obtained from Janiver Labs (Le Genest-Saint-Isle, France) and studies conducted by Oncodesign® Biotechnology (Dijon Cedex, France). For tolerance experiments, animals (3 per group) received escalating doses of P604 ACE2 peptide inhibitor or ACE2i peptide via intraperitoneal injection. Doses were escalated daily (day 1: 25 mg/kg, day 2: 50 mg/kg, day 3: 100 mg/kg) and animals monitored prior to culling on day 4. Animal viability, behaviour and rectal temperature were recorded every 2 hours over a 6 hour period post-administration and body weights were measured daily. For P604 ACE2 peptide inhibitor efficacy studies, animals (8 per group) were treated with vehicle (IP, daily day 0, 1 and 2) (Sodium chloride 0.9%, Osalia, Paris, France) or P604 ACE2 peptide inhibitor over a 2-day period via intravenous (IV, 15 mg/kg, day 0 once and day 1 twice, 8 hours apart) or intraperitoneal (IP, 100 mg/kg, daily Day 0, 1, 2) injection. For P604 ACE2 peptide inhibitor efficacy studies, animals were treated with vehicle or P604 ACE2 peptide inhibitor (30 mg/kg, IN, twice daily, Day 0, 1 8 hours apart).

For all efficacy studies, peptides were administered to animals 1 hr prior to SARS-Cov-2 infection on Day 0 (10⁴ PFU; IN administration) with the SARS-CoV-2 strain “Slovakia/SK-BMC5/2020”, originally provided by the European Virus Archive global. All procedures on golden Syrian Hamsters were submitted to the Institutional Animal Care and Use Committee of CEA approved by French authorities.

### Virus Load Determination in Lunas by Aenomic RT-ciPCR.

Quantification of lung viral load by RT-qPCR was performed using the viral ORF1 ab gene (Fwd: CCGCAAGGTTCTTCTTCGTAAG [SEQ ID NO: 45], Rvs: TGCTATGTTTAGTGTTCCAGTTTTC [SEQ ID NO: 46], Probe: AAGGATCAGTGCCAAGCTCGTCGCC [5′] Hex [3′] BHQ-1 [SEQ ID NO: 47]). Extraction of viral RNA was conducted using the NucleoSpin® 96 Virus Core Kit (Macherey Nagel, Duren, Germany) and frozen at −80° C. until qRT-PCR. Complete qRT-PCR was run using SuperScript™ III One-Step qRT-PCR System kit (catalogue #1732020, Life Technologies, Carlsbad, CA) with primers and qRT-PCR conditions targeting ORF1ab gene. Amplifications were performed using a Bio-Rad CFX384TM (Bio-Rad, Hercules, CA) and adjoining software.

### Virus TCID50 Determination in Lungs.

Two hours before testing, Vero E6/TMPRSS2 cells were plated in 96-well plates at the density of 25,000 cells per well in a volume of 200 μL of complete growth medium (DMEM 10% FCS). Cells were infected with serial dilutions of the day 2 lung homogenate (triplicate) for 1 h at 37° C. Fresh medium was then added for 72 hours. After cell infection, an MTS/PMS assay was performed according to provider protocol (Cat #G5430, Promega, Madison, WI). Briefly, after discarding 100 pL of supernatant, a volume of 20 μL of MTS/PMS reagent was added to the remaining 100 pL supernatant. After 4 hours, plates were read using an Elisa Plate reader and data recorded.

### Example 14

### P604 ACE2 Peptide Induces Anti-Viral Signature & Abroqates Nuclear ACE2

The present inventors next wanted to address the presence of CD3-positive T lymphocytes, as previous studies have shown that CD3+ T lymphocytes were detected in the peribronchial region at 5 dpi, which may facilitate the rapid clearance of the infected cells.

Treatment with the P604 ACE2 peptide inhibitor via either IP or IV routes was able to significantly induce higher cells positive for perforin and induce more CD3+ cells to express perforin. Treatment with the P604 ACE2 peptide inhibitor significantly enhanced expression of effector marker perforin which is essential for anti-viral activity. Overall, the P604 ACE2 peptide inhibitor is able to induce a strong anti-viral signature via the increase of perforin and CD3 infiltration.

### Materials & Methods

### Immunofluorescence

IFA imaging and analysis was carried out using previously established and optimized protocols. Cells were fixed with formaldehyde (3.7%) and then immuno-stained with antibodies targeting the SARS-CoV-2 viral spike, custom antibodies ACE2me1, ACE2unmod. Cells were permeabilized by incubating with 0.5% Triton X-100 for 15 min, blocked with 1% BSA in PBS, and were probed with primary antibodies followed by visualization with secondary donkey anti-rabbit, mouse, or goat antibodies conjugated to Alexa Fluor 488, 568, or 647. Coverslips were mounted on glass microscope slides with ProLong Glass Antifade reagent (Life Technologies, Carlsbad, CA). Protein targets were localized by digital pathology laser scanning microscopy. Single 0.5 pm sections were obtained using an ASI Digital pathology (ASI Digital pathology is characterization of both the fluorescent intensity as per normal immunofluorescent imaging as well as the ability to count the population of cells positive or negative for antibodies, allow population dynamics to be investigation using powerful custom designed algorithms and automated stage. This also allows the imaging and counting of large cell numbers for statistical power) microscope using a 100× oil immersion lens running ASI software. The final image was obtained by averaging four sequential images of the same section. Digital images were analyzed using automated ASI software as described previously (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analyzed using ImageJ software (ImageJ, NIH, Bethesda, MD, USA) to determine the total cell fluorescence or cell surface only fluorescence for non-permeabilized cells. Appropriate controls were used for all experiments including no antibody controls, primary only, or secondary only controls.

Opal Tyramide staining, unlike traditional IFA allows the use of antibodies from the same host species. Imaging and analysis was carried out using previously established and optimized protocols for permeabilization and antigen retrieval. All FFPE sections were stained with Opal Tyramide staining. Samples were dewaxed using a decloaking chamber and were prepared using either 0.1% Triton X-100 20 min, Biocare Medical Denaturing Solution, or Dako pH6.0/pH 9.0 for antigen retrieval. Sniper+BSA was used for blocking (10 minutes). Primary antibodies employed include CD3, Perforin, SARS-CoV-2 Spike and custom antibody ACE2me1 with VGY or DVG buffers. Primary antibodies were detected with MACH2 HRP secondary with Opal fluorochromes 520, 570 or 650. Imaging and analysis was then carried out as per Immunofluorescent staining and analysis described above using the ASI Digital Pathology platform for automated counting and intensity analysis.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

### REFERENCES

Kosugi, S., Hasebe, M., Entani, T., Takayama, S., Tomita, M., and Yanagawa, H. (2008). Design of peptide inhibitors for the importin alpha/beta nuclear import pathway by activity-based profiling. Chemistry & biology 15, 940-949.
Kosugi, S., Hasebe, M., Matsumura, N., Takashima, H., Miyamoto-Sato, E., Tomita, M., and Yanagawa, H. (2009). Six classes of nuclear localization signals specific to different binding grooves of importin alpha. The Journal of biological chemistry 284, 478-485.
Kosugi, S., Hasebe, M., Tomita, M., and Yanagawa, H. (2009). Systematic identification of cell cycle-dependent yeast nucleocytoplasmic shuttling proteins by prediction of composite motifs. Proceedings of the National Academy of Sciences of the United States of America 106, 10171-10176.
Wen, P. P., Shi, S. P., Xu, H. D., Wang, L. N., and Qiu, J. D. (2016). Accurate in silico prediction of species-specific methylation sites based on information gain feature optimization. Bioinformatics (Oxford, England) 32, 3107-3115.
Blom, N., Sicheritz-Ponten, T., Gupta, R., Gammeltoft, S., and Brunak, S. (2004). Prediction of post-translational glycosylation and phosphorylation of proteins from the amino acid sequence. Proteomics 4, 1633-1649.
Su, H., Liu, M., Sun, S., Peng, Z., and Yang, J. (2019). Improving the prediction of protein-nucleic acids binding residues via multiple sequence profiles and the consensus of complementary methods. Bioinformatics (Oxford, England) 35, 930-936.
Ofran, Y., and Rost, B. (2007). ISIS: interaction sites identified from sequence. Bioinformatics (Oxford, England) 23, e13-16.
Huang, J., Sengupta, R., Espejo, A. B., Lee, M. G., Dorsey, J. A., Richter, M., Opravil, S., Shiekhattar, R., Bedford, M. T., Jenuwein, T., and Berger, S. L. (2007). p53 is regulated by the lysine demethylase LSD1. Nature 449, 105-108.
Sheng, W. et al. LSD1 Ablation Stimulates Anti-tumor Immunity and Enables Checkpoint Blockade. Cell 174, 549-563 e519, doi:10.1016/j.ce11.2018.05.052 (2018).
Shang, J. et al. Structural basis of receptor recognition by SARS-CoV-2. Nature 581, 221224, doi:10.1038/s41586-020-2179-y (2020).

1. An isolated or purified proteinaceous molecule, which comprises or consists essentially of an amino acid sequence represented by Formula I [SEQ ID NO: 13]:
(Formula I)
TGIRDRX₁X₂X₃NKARS
wherein X₁, X₂, and X₃ are independently selected from K, A, and Q amino acids, or modified forms thereof.

2. The proteinaceous molecule of claim 1, wherein X₁ is a lysine (K) residue and/or wherein X₂ is a lysine (K) residue, and/or wherein X₃ is a lysine (K) residue.

3. (canceled)

4. (canceled)

5. (canceled)

6. The proteinaceous molecule according to claim 1, wherein each of X₁, X₂, and X₃ are acetylated K residues or methylated K residues.

7. (canceled)

8. The proteinaceous molecule according to claim 1, which is represented by Formula II [SEQ ID NO: 17]:
(Formula II)
Z₁TGIRDRX₁X₂X₃NKARSZ₂
wherein X₁, X₂, and X₃ are as broadly defined above;
Z₁ is absent or is selected from at least one of a proteinaceous moiety comprising from about 1 to about 50 amino acid residues, and a protecting moiety; and
Z₂ is absent or is selected from at least one of a proteinaceous moiety comprising from about 1 to about 50 amino acid residues.

9. The proteinaceous molecule according to claim 8 wherein Z₁
comprises an amino acid sequence represented b Formula III:
(Formula III)
B₁X₄X₅X₆
wherein:
B₁ is absent or is an N-terminal blocking residue;
X4 is absent or is selected from any amino acid;
X5 is absent or is selected from any amino acid; and
X6 is absent or is selected from any amino acid.

10. The proteinaceous molecule according to claim 1, wherein the molecule comprises, consists, or consists essentially of and/or shares at least 80% sequence identity with an amino acid sequence:
[SEQ ID NO: 12]
TGIRDRKKKNKARSGENPYASIDISKGENNPGFQNTDDVQTSF.

11. (canceled)

12. (canceled)

13. An isolated or purified proteinaceous molecule, which comprises or consists essentially of an amino acid sequence that corresponds to a SARS-CoV-2 ACE-2 amino acid sequence, wherein the amino acid sequence includes a lysine residue corresponding to Lys31 of the native human ACE-2 amino acid sequence, optionally wherein the proteinaceous molecule comprising, or consisting, or consisting essentially of the amino acid sequence IEEQAKTFLDK [SEQ ID NO: 23] or comprising a peptide with an amino acid sequence represented by Formula IV [SEQ ID NO: 23]:
(Formula IV)
Z₁IEEQAKTFLDKZ₂
wherein:
Z₁ is absent or is selected from at least one of a proteinaceous moiety comprising from about 1 to about 50 amino acid residues, and a protecting moiety; and
Z₂ is absent or is selected from at least one of a proteinaceous moiety comprising from about 1 to about 50 amino acid residues.

14. (canceled)

15. (canceled)

16. The proteinaceous molecule of claim 13, wherein Z₁ is absent, and Z₂ comprises an amino acid sequence of FNHEAEDLFYQSSLASWNYNT and/or wherein the proteinaceous molecule comprising or consisting, or consisting essentially of the amino acid sequence:
[SEQ ID NO: 24]
IEEQAKTFLDKFNHEAEDLFYQSSLASWNYNT.

17. (canceled)

18. The proteinaceous molecule of any one of claim 13, wherein Z₁ comprises the amino acid sequence ST, and Z₂ is absent, and/or the proteinaceous molecule comprising, consisting, or consisting essentially of the amino acid sequence STIEEQAKTFLDK [SEQ ID NO: 26].

19. (canceled)

20. A composition for treating or preventing a coronavirus infection or reducing SARS-CoV replication in a cell, comprising an agent selected from a proteinaceous molecule and a pharmaceutically acceptable carrier or diluent, wherein the proteinaceous molecule is defined in claim 1.

21. (canceled)

22. (canceled)

23. (canceled)

24. (canceled)

25. (canceled)

26. (canceled)

27. The proteinaceous molecule of claim 1, further comprising at least one anti-viral agent.

28. (canceled)

29. A method for reducing SARS-CoV replication in a cell, the method selected from a proteinaceous molecule according to claim 1 for a time and under conditions sufficient to reduce coronavirus entry in the cell.

30. (canceled)

31. (canceled)

32. (canceled)

33. (canceled)

34. (canceled)

35. A method for preventing or reducing SARS-CoV entry into a cell of a subject, the method comprising administering to the subject an effective amount of an agent selected from the proteinaceous molecule according to claim 1.

36. (canceled)

37. (canceled)

38. (canceled)

39. A proteinaceous molecule, comprising, consisting, or consisting essentially of, an amino acid sequence corresponding to the C-terminal tail region of an ACE-2 protein, optionally wherein the molecule comprises less than 50 amino acid residues, or less than 25 amino acid residues, or less than 15 amino acid residues, optionally wherein the amino acid sequence comprises a nuclear localization sequence.

40. (canceled)

41. (canceled)

42. (canceled)

43. (canceled)

44. The proteinaceous molecule of claim 39, wherein the C-terminal tail region corresponds to at least residues 763 to 805 of the wild-type human ACE-2 protein (as set forth in SEQ ID NO:1).

45. The proteinaceous molecule of claim 1, wherein the proteinaceous composition comprises a protecting moiety, optionally Myr.

46. (canceled)

47. (canceled)

48. (canceled)

49. (canceled)

50. (canceled)

51. (canceled)

52. (canceled)

53. (canceled)

54. (canceled)

55. The composition of claim 20, wherein the coronavirus infection is a SARS-CoV infection.
